(19) United States
(12) Patent Application Publication
(10) Pub. No.: US 2021/0022717 A1
(43) Pub. Date: Jan. 28, 2021

(54) FOLLICULAR CONTENT STORAGE

(71) Applicant: Carmel Diagnostics Ltd., Kiryat-Tivon (IL)

(72) Inventors: Bezalel CNAANI, Kiryat-Tivon (IL); Amnon WEICHSELBAUM, Haifa (IL)

(73) Assignee: Carmel Diagnostics Ltd., Kiryat-Tivon (IL)

(21) Appl. No.: 17/041,046

(22) PCT Filed: Mar. 26, 2019
(86) PCT No.: PCT/IL2019/050344

§ 371 Date: Sep. 24, 2020

Related U.S. Application Data
(60) Provisional application No. 62/647,849, filed on Mar. 26, 2018.

(51) Int. Cl. A61B 10/02; A61B 10/00

(52) U.S. Cl. CPC A61B 10/0291; A61B 10/0096; A61B 10/0283

(57) ABSTRACT

A method for isolating and storing follicular content, including:
repeatedly collecting follicular content including an oocyte and follicular fluid (FF) from a plurality of follicles of a female subject during a single oocyte retrieval process;
storing each oocyte and follicular fluid originating from the same follicle in a separate storage compartment from the rest of the collected follicular content.

### RELATED APPLICATION

This application claims the benefit of priority under 35 USC § 119(e) of U.S. Provisional Patent Application No. 62/647,849 filed 26 Mar. 2018, the contents of which are incorporated herein by reference in their entirety.

### FIELD AND BACKGROUND OF THE INVENTION

The present invention, in some embodiments thereof, relates to follicular content isolation and, more particularly, but not exclusively, to follicular content isolation and storage.

### SUMMARY OF THE INVENTION

Some examples of some embodiments of the invention are listed below:

Example 1. A method for isolating and storing follicular content, comprising:
repeatedly collecting follicular content comprising an oocyte and follicular fluid (FF) from a plurality of follicles of a female subject during a single oocyte retrieval process;
storing each oocyte and follicular fluid originating from the same follicle in a separate storage compartment from the rest of said collected follicular content.
Example 2. The method of example 1, comprising:
isolating a follicular fluid sample from said stored follicular fluid;
associating said follicular fluid sample with a specific oocyte.
Example 3. The method of example 2 comprising tagging said FF sample and said oocyte with tags, and wherein said associating comprises matching said FF sample with said oocyte based on said tags.
Example 4. The method of any one of the examples 2 and 3, wherein said storing comprises storing said follicular content from each follicle of said plurality of follicles in according to a pre-determined order, and wherein said associating comprises associating said FF sample with said oocyte based on said order.
Example 5. The method of any one of examples 2 to 4, wherein a volume of said FF sample is at least 10 microliters.
Example 6. The method of any one of examples 2 to 5, comprising analyzing said FF sample to determine at least one parameter related to a state of said associated oocyte.
Example 7. The method of example 6, wherein said at least one parameter comprises oxidation levels.
Example 8. The method of any one of examples 6 or 7, wherein said at least one parameter is selected from a list comprising of vitality level, maturation level ability of the oocyte to be fertilized, cleavage rates, nitric oxide levels, malondialdehyde levels, and reduced glutathione levels.
Example 9. The method of any one of examples 6 to 8 comprising selecting an oocyte for an IVF process based on results of said FF sample analysis.
Example 10. The method of any one of examples 6 to 9, comprising cryopreserving at least some isolated oocytes from said plurality of follicles based on results of said FF sample analysis.
Example 11. A system for sorting follicle content, comprising:
a storage assembly comprising a plurality of storage compartments;
a first fluid flow path between a needle at least partly insertable into a follicle and a first storage compartment of said plurality of storage compartments;
at least one movable portion comprising an actuator configured to individually align a distal opening of said first fluid flow path with a different storage compartment of said storage assembly;
a control unit, comprising:
a control circuitry electrically connected to said actuator and configured to signal said actuator to individually align said distal opening with a different storage compartment of said storage assembly.
   Example 12. The system of example 11, comprising an interface configured to deliver a human detectable indication related to alignment of said distal opening with at least one storage compartment of said plurality of said storage compartments.
   Example 13. The system of example 12, wherein said human detectable indication comprises a sound and/or a visible indication.
   Example 14. The system of any one of examples 12 or 13, wherein said movable portion comprises a movable storage compartment cover connected to said distal opening of said first fluid flow path, and is configured to move said distal opening relative to a selected storage compartment of said plurality of said storage compartments in response to a signal received from said control circuitry.
   Example 15. The system of any one of examples 12 to 14, wherein said movable portion comprises a movable storage assembly, and is configured to move said storage assembly relative to said distal opening in response to a signal received from said control circuitry.
   Example 16. The system of any one of examples 12 to 15, comprising at least one sensor on said first flow path electrically connected to said control circuitry, wherein said control circuitry is configured to signal said movable portion to align said distal opening with said different storage compartment of said storage assembly based on signals received from said at least one sensor.
   Example 17. The system of example 16, wherein said at least one sensor comprises a flow sensor on said first fluid flow path, configured to sense follicular content flow or changes in follicular fluid flow within said first flow path.
   Example 18. The system of any one of examples 16 or 17, wherein said at least one sensor comprises a pressure sensor on said first fluid flow path configured to measure pressure levels and/or changes in pressure levels within said first fluid flow path.
   Example 19. The system of any one of examples 16 to 18, wherein said at least one sensor comprises an optic sensor, configured to detect flow and/or follicular content within said first fluid flow path based on at least one optical parameter related to said follicular content.
   Example 20. The system of any one of examples 12 to 19, comprising at least one optical sensor on said storage assembly electrically connected to said control circuitry, wherein said at least one optical sensor is configured to sense at least one optical parameter of follicular content in at least one storage compartment of said storage assembly.
   Example 21. The system of any one of examples 12 to 20, comprising a first negative pressure source functionally connected to said first fluid flow path, and configured to generate negative pressure within said first fluid flow path.

Example 22. The system of example 21, wherein said control circuitry signals said interface to generate said human detectable indication when said first negative pressure source is activated.

Example 23. The system of example 21, wherein said interface comprises a user input element configured to receive sensory input from a user of said system, and wherein said first negative pressure source is activated based on signals received from said user input element.
Example 24. The system of example 23, wherein said user input element comprises a foot switch or a button.
Example 25. The system of any one of examples 16 to 24 comprising a second fluid flow path connecting said first fluid flow path and at least one follicular fluid (FF) collecting compartment.
Example 26. The system of example 25, wherein said FF collecting compartment comprises an optical analyzer cuvette.
Example 27. The system of any one of examples 25 or 26, comprising a second negative pressure source functionally connected to said second fluid flow path and electrically connected to said control circuitry, wherein said second negative pressure source is configured to generate negative pressure within said second fluid flow path in response to electrical signals received from said control circuitry.
Example 28. The system of example 27, wherein said control circuitry is configured to signal said second negative pressure source to generate said negative pressure within said second flow path for a time duration sufficient to deliver at least 50 μl of FF into said FF collecting compartment.
Example 29. The system of any one of examples 27 or 28, wherein said control circuitry signals said second negative pressure source to generate said negative pressure within said second fluid flow path based on signals received from said at least one sensor on said first fluid flow path.
Example 30. The system of any one of examples 25 to 29, comprising a filter positioned within said second fluid flow path, wherein said filter comprises a plurality of pores shaped and sized to prevent passage of an oocyte from said first fluid flow path into said second fluid flow path.
Example 31. The system of example 30, wherein a largest dimension of said pores is in a range of 2-50 micron.
Example 32. The system of any one of examples 25 to 31, comprising a washing fluid source fluidically connected to said second fluid flow path and/or to said first fluid flow path by a valve electrically connected to said control circuitry.
Example 33. The system of example 32, wherein said control circuitry is configured to open said valve before the alignment of said first fluid flow path with said different storage compartment and/or before collecting a second FF sample through said second fluid flow path.
Example 34. An oocyte filter, comprising:
a hollow tube with an inner lumen, shaped and sized to be positioned within a fluid flow path connected to a needle configured to deliver an oocyte and follicular fluid between a follicle and a storage compartment, wherein said hollow tube comprises at least one surface dividing said inner lumen into two isolated chambers,
wherein said at least one surface comprises a plurality of pores shaped and sized to prevent passage of said follicle through said pores.
Example 35. The filter of example 34, wherein a largest dimension of said pores is in a range of 2-50 micron.
Example 36. A needle assembly, comprising:
an elongated hollow needle having a distal end shaped and sized to penetrate into a follicle, and a proximal end;
at least one sensor mounted on or within the needle and configured to sense at least one parameter related to flow of follicular content within a lumen of said needle.
Example 37. The needle of example 36, wherein said at least one sensor comprises a pressure sensor configured to sense pressure levels and/or changes in pressure levels within said lumen.
Example 38. The needle of any one of examples 36 or 37, wherein said at least one sensor comprises a flow sensor, configured to sense flow levels and/or changes in flow levels within said lumen.
Example 39. The needle of any one of examples 36 to 38, comprising:
a battery connected to the outer surface of said hollow needle;
a control circuitry electrically connected to said battery and said at least one sensor, configured to measure said at least one parameter related to flow of said follicular content within said lumen of said needle based on signals received from said at least one sensor.
Example 40. The needle of example 39, comprising:
an interface electrically connected to said control circuitry, configured to deliver a human detectable indication;
wherein said control circuitry signals said interface to generate said human detectable indication based on signals received from said at least one sensor.
Example 41. The needle of example 40, wherein said control circuitry signals said interface to generate said human detectable indication when pressure levels within said lumen are lower than a predetermined value.
Example 42. The needle of any one of examples 40 or 41, wherein said control circuitry signals said interface to generate said human detectable indication when flow levels within said lumen are lower than a predetermined value.
Example 43. The needle of any one of examples 40 to 42, wherein said follicular content comprises an oocyte and/or follicular fluid.
Example 44. A method for generating a database, comprising:
providing data on each oocyte of a plurality of oocytes using an input element, wherein each of said oocytes is isolated separately from the same female subject during a single oocyte retrieval process and wherein each of said oocytes is stored separately from the rest of said oocytes; inserting said data on each oocyte into a separate entry in a database stored in a memory electrically connected to said input element.
Example 45. The method of example 44, wherein said data comprises results of follicular fluid analysis, wherein said follicular fluid is isolated from the same follicle as a corresponding oocyte in said database.
Example 46. The method of example 45, wherein said data comprises an indication related to oxidative levels of said follicular fluid.
Example 47. The method of any one of examples 44 to 46, wherein said data comprises at least one indication related to the ability of an oocyte in said database to be fertilized in an in-vitro fertilization process.
Example 48. The method of any one of examples 44 to 47, wherein said data comprises at least one indication related to the ability of an oocyte in said database to be cryopreserved.
Example 49. The method of any one of examples 44 to 48, comprising applying at least one algorithm stored in said memory for selecting an entry related to a single oocyte or a group of entries related to a plurality of oocytes, according to at least one parameter related to said data.
Example 50. A method for scoring an oocyte, comprising:
providing a database comprising a plurality of oocytes entries and data derived from follicular fluid analysis individually associated with each oocyte of said plurality of oocytes;
scoring at least some of said oocytes based on said follicular fluid analysis data.
Example 51. The method of example 50, comprising:
selecting an oocyte for cryopreservation and/or in vitro fertilization based on said scoring.
Example 52. The method of example 50, comprising:
selecting an embryo for cryopreservation based on said scoring.
Example 53. The method of example 50, comprising:
selecting an embryo to be transferred back into the uterus based on said scoring.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as determining changes in pressure or flow levels within an isolation flow path, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a flow chart of an IVF and/or an Oocyte cryopreservation process including isolation of FF of each Oocyte, according to some embodiments of the invention;

FIG. 1B is a flow chart of a separate process for isolation and storage of follicular content from each follicle, according to some embodiments of the invention;

FIG. 2 is a block diagram of a system for isolation and storage of the follicular content from each follicle separately from the content of other follicles, according to some embodiments of the invention;

FIG. 3 is a flow chart of a process for identifying an empty follicle, according to some embodiments of the invention;

FIG. 4A is a schematic illustration of a needle with sensors for isolation of follicular content, according to some embodiments of the invention;

FIGS. 4B and 4C are schematic illustrations of a needle penetrating into a follicle, according to some embodiments of the invention;

FIG. 4D is a graph describing changes in flow when sequentially penetrating into a plurality of follicles, according to some embodiments of the invention;

FIG. 4E is a schematic illustration of a tube squeezer, according to some embodiments of the invention;

FIG. 5A is a flow chart of a process for separating an Oocyte from its FF, according to some embodiments of the invention;

FIG. 5B is a system for separating an Oocyte from its FF into a different storage compartment, according to some embodiments of the invention;

FIGS. 5C-5E are schematic illustration of a FF filter, according to some embodiments of the invention;

FIG. 5F is a FF filter on a follicular content isolation flow path, according to some embodiments of the invention;

FIG. 6 is a schematic illustration of a system for separately isolation and storage of the follicular content from each follicle, according to some embodiments of the invention;

FIG. 7A is a schematic illustration of a fluid distribution system, according to some embodiments of the invention;

FIG. 7B is a flow chart of a process for replacing a storage compartment when isolating the content of a new follicle, according to some embodiments of the invention;

FIGS. 8A-8C are schematic illustrations of a sealed connection between a vacuum sealing cap and a storage compartment, according to some embodiments of the invention;

FIGS. 9A-9E are schematic illustrations of a linear storage compartments cartridge, according to some embodiments of the invention;

FIGS. 10A-10C are schematic illustrations of a round storage compartments cartridge, according to some embodiments of the invention;

FIGS. 11A-11D are schematic illustrations of a mechanism for removal of a vacuum sealing cap, according to some embodiments of the invention;

FIGS. 12A-12D are schematic illustration of a mechanism for removal of a vacuum sealing cap when moving in XYZ axis, according to some embodiments of the invention; and

FIGS. 13A-13C are schematic illustrations of an optical assembly of a system for isolating and storage the content of a follicle, according to some embodiments of the invention.

### DESCRIPTION OF SPECIFIC EMBODIMENTS OF THE INVENTION

The present invention, in some embodiments thereof, relates to follicular content isolation and, more particularly, but not exclusively, to follicular content isolation and storage.

A broad aspect of some embodiments relates to matching separately isolated a follicular fluid (FF) sample with a specific oocyte. In some embodiments, the FF sample and the oocyte are isolated from the same follicle, for example during an oocyte retrieval process. In some embodiments, the FF and the oocyte are stored together in the same storage compartment, separately from the rest of the oocytes. Alternatively, the FF sample is stored separately from the oocyte.

According to some embodiments, isolated oocytes are organized in a specific order, and a FF sample is matched to a specific oocyte based on the order. Alternatively or additionally, each of the isolated oocytes is tagged and the FF sample is matched to a specific oocyte based on the oocyte tag, optionally a tag that matches a tag of the FF sample.

According to some embodiments, a FF sample is collected from a storage compartment of FF and oocyte, and analyzed, for example to determine at least one parameter related to the state or the condition of the oocyte. In some embodiments, the FF sample is stored separately and analyzed to determine the at least one parameter related to the state or the condition of the oocyte. Optionally, the FF sample is analyzed during the isolation process, for example by an optical assembly.

An aspect of some embodiments relates to separately storing each Oocyte with her own follicular fluid (FF) during Oocyte retrieval from a plurality of follicles. In some embodiments, the follicle content of each follicle which comprises an Oocyte and the Oocyte's FF is directed to a new storage compartment, for example a vial, a tube, a plate, a well of a tissue culture wells plate, or a cuvette. In some embodiments, when penetrating into a new follicle, for example to isolate the follicle content, the storage compartment is replaced optionally automatically with a new storage compartment. Alternatively, when penetrating into a new follicle, a fluid flow path is diverted to a new storage compartment.

According to some embodiments, the follicle content is retrieved by application of suction forces through a hollow needle tip positioned inside the follicle, optionally by vacuum. In some embodiments, when the follicle is empty, changes in pressure within a fluid flow path connected to the needle are detected. Optionally, in response to the pressure changes the storage compartment is replaced or the fluid flow path is diverted to a new storage compartment.

According to some embodiments, a sensor positioned on the fluid flow path, for example an optic sensor, is configured to sense the presence of an Oocyte and optionally to signal a control unit of the system to stop the suction forces application and/or to provide an indication to a user of the system. Alternatively or additionally, a sensor, for example an optic sensor is positioned at the storage compartment and is configured to determine the presence of an Oocyte within the storage compartment. Optionally, the storage compartment sensor is configured to signal a control unit of the system to stop the suction forces application and/or to deliver an indication to a user of the system.

According to some embodiments, the separately isolated and optionally stored FF of each Oocyte is analyzed, for example to determine at least one parameter of the Oocyte, for example the vitality level, oxidation level, maturation level, ability of the Oocyte to be fertilized, cleavage rates, nitric oxide (NO) levels, malondialdehyde (MDA) levels and reduced glutathione (GSH), or any indicator of the oxidative status of the follicle or the oocyte, for example to predict the outcome of in vitro fertilization. Optionally, the FF is analyzed, for example as described in Wiener-Megnazi Z. et al. Fertil Steril. 2004 October, and as described in Ender Yalçinkaya et al. J Turk Ger Gynecol Assoc. 2013 September

In some embodiments, an Oocyte is selected for in-vitro fertilization based on the results of the analysis.

According to some embodiments of the invention, each oocyte is stored with her own FF, both isolated from the same follicle, for example to prevent contamination with FF from other follicles. In some embodiments, the allowed cross contamination level with FF from a different follicle is up to 10% of the total volume, for example 10%, 8%, 5% or any intermediate, smaller or larger percentage of contamination of the total volume.

An aspect of some embodiments relates to separately collecting at least some of the FF of each Oocyte during Oocyte retrieval. In some embodiments, the FF of a specific Oocyte is stored separately from the Oocyte and from FF of other Oocytes. In some embodiments, the FF is separated from the Oocyte during Oocyte retrieval, for example by diverting at least some of the FF to a different fluid flow path than the flow path used for the isolation of the Oocyte.

According to some embodiments, the follicle content passes through a semi-permeable filter that allows passing of fluid and particles smaller than 60 microns, for example 60 microns, 30 microns, 25 microns, 20 microns, 10 microns or any intermediate, smaller or larger value. In some embodiments, the particles and/or fluid which are smaller than 60 micron pass the filter towards a FF storage compartment, for example a tube, a vial, or a cuvette. Optionally, the particles and/or fluid pass the 60-micro filter towards a filter with smaller pores, for example towards a filter with pores smaller than 20 microns, for example 20 microns, 10 microns, 5 microns or any intermediate, smaller or larger value. In some embodiments, particles and/or fluid pass through the second filter, for example to filter out different cell types and debris, for example to filter out red blood cells, epithelial cells, from the isolated FF sample. In some embodiments, particles larger than 60 microns, for example 60 microns, 70 microns, 80 microns, 100 microns, or any intermediate, smaller or larger value are diverted to a different storage compartment, optionally an Oocyte storage compartment. Optionally, the particles which are larger than 60 microns comprise an Oocyte.

According to some embodiments, the FF is analyzed, for example as described above, to determine at least one parameter of the Oocyte. In some embodiments, when the Oocyte is planned to be cryopreserved, the at least one parameter is related to the ability of the Oocyte to remain viable after a thawing process. Additionally or alternatively, the at least one parameter is related to the ability of the Oocyte to be fertilized, optionally in-vitro fertilized after a thawing process.

An aspect of some embodiments relates to aligning a new storage compartment with a follicular content isolation flow path when isolating the content of a new follicle. In some embodiments, a control unit of a system for Oocyte retrieval aligns a new storage compartment to collect the content of a new follicle when penetrating into a new follicle. Alternatively, the control unit aligns a new storage compartment when detecting that a follicle is empty. Optionally, the control unit aligns a new storage compartment, when receiving a signal from a user of the system.

According to some embodiments, the control unit aligns a new storage compartment with a follicular isolation flow path, for example when detecting the presence of an Oocyte within the storage compartment and when detecting a sufficient amount of FF. In some embodiments, a sufficient amount of FF is the amount needed for analysis of FF, storing FF, for example for later analysis and/or freezing FF, for example for later analysis. Optionally a sufficient amount of FF is at least 50 microliter (μl), for example 50 μl, 100 μl, 200 μl, 500 μl or any intermediate, smaller or larger value.

According to some embodiments, the new storage compartment is aligned with the isolation flow path by connecting a new storage compartment to a proximal opening of the flow path. In some embodiments, the proximal opening of the flow path is stationary and the new storage compartment is forwarded to the opening. Alternatively, the new storage compartment is stationary and the proximal opening is advanced towards the new storage compartment, for example by a movable arm or a movable frame mechanically connected to the proximal opening.

According to some exemplary embodiments, a flow path comprises a movable portion. In some embodiments, the distal opening of the flow path is connected to the movable portion. Optionally, the movable portion is a cover of a storage compartment. In some embodiments, the movable portion comprises an actuator, for example a motor. In some embodiments, the control circuitry signals the motor to move the cover to a new storage compartment, for example before inserting a needle into a new follicle.

According to some exemplary embodiments, a storage assembly which includes a plurality of storage compartment, for example tubes and/or vials is a movable storage assembly and optionally includes a movable portion. In some embodiments, the control circuitry signals an actuator connected to the movable storage assembly to align a storage compartment of the plurality of storage compartments with a distal opening of the flow path. Alternatively, the control circuitry is configured to signal a first actuator connected to the cover and a second actuator connected to the storage assembly to align a distal opening of the flow path with a storage compartment positioned in the storage assembly.

An aspect of some embodiments relates to clearing residual content of a follicular content isolation flow path before isolating the content of a new follicle. In some embodiments, the isolation flow path is cleared before or when a hollow needle of a follicular content isolation system penetrates into a new follicle. Optionally, the isolation flow path is cleared when identifying that the follicle is empty.

According to some embodiments, the isolation flow path is cleared by passing fluid through flow path, for example air or liquid. In some embodiments, the liquid comprises a clearing solution. In some embodiments, the isolation flow path is cleared automatically by a control unit of the isolation system. Alternatively, the isolation flow path is cleared when the control unit receives a signal from a user of the system. Optionally, the isolation path is manually cleared by a user, for example when receiving an indication that the follicle is empty and/or that the hollow needle exits the follicle. In some embodiments, isolation flow path is cleared when the isolation process is stopped and/or that suction forces applied, for example to remove the content of the follicle are stopped.

According to some embodiments, the flow path is cleared to prevent contamination with residual FF from a different follicle. In some embodiments, the maximal allowed cross contamination level with FF from a different follicle is up to 10% of the total volume, for example 10%, 8%, 5% or any intermediate, smaller or larger percentage of contamination of the total volume.

An aspect of some embodiments relates to generating a new sealed flow path for vacuum-isolated follicle content when isolating the content of a new follicle. In some embodiments, a sealing element is connected to a proximal opening of a follicle content isolation flow path. In some embodiments, the sealing element is configured to seal a connection between the flow path and a storage compartment, for example a vial, a tube or a cuvette. Optionally, the sealing element comprises a sealing cap or a sealing cover shaped and sized to fit an opening of the storage compartment.

According to some embodiments, the sealing element is configured to re-attach and disconnect from the storage compartment. In some embodiments, the sealing element is pressed against the storage compartment opening with sufficient force to allow, for example sealing of a fluid path between the isolation fluid flow and the storage compartment. Alternatively or additionally, the storage compartment opening is pressed against the sealing element with sufficient force to allow, for example sealing of a fluid path between the isolation fluid flow and the storage compartment. Optionally, the sealing is interrupted when the sealing element is pulled away from the storage compartment opening, and/or when the storage compartment opening is pulled away from the sealing element. Optionally, the sealing is interrupted when replacing a storage compartment or during flushing or cleaning the isolation flow path.

An aspect of some embodiments relates to generating a database which includes oocytes entries and data and/or indications of data derived from FF analysis associated with each of the oocytes. In some embodiments, the FF analysis is performed on FF collected with a specific oocyte from the same follicle. In some embodiments, at least some of the oocytes are scored based on the FF analysis data. In some embodiments, at least one oocyte is selected for in-vitro fertilization based on the data. In some embodiments, at least one oocyte is selected for cryopreservation based on the data. In some embodiments, at least one embryo is selected to be returned to the uterus of a subject based on the data.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Exemplary Process for IVF and/or Oocytes Cryopreservation

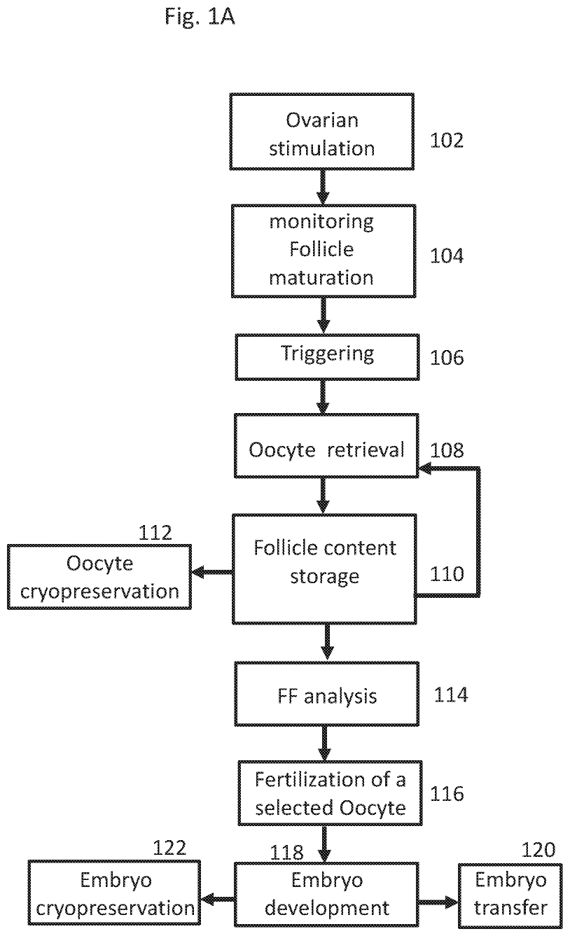

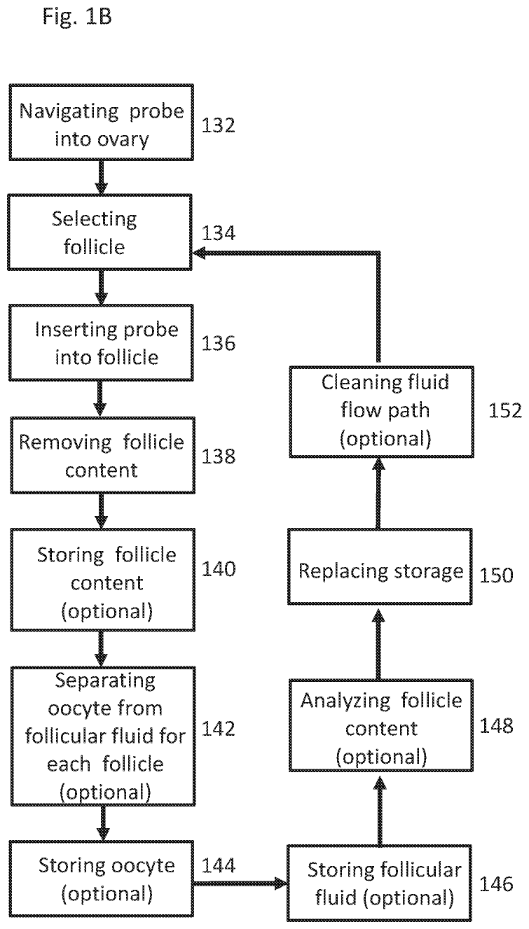

According to some exemplary embodiments, the content of a single follicle is stored separately from the content of other follicles, as part of an IVF process and/or as part of an Oocytes cryopreservation process. Reference is now made to FIG. 1A depicting a process for IVF or Oocytes cryopreservation, according to some exemplary embodiments of the invention.

According to some exemplary embodiments, the ovaries of a female subject are stimulated at 102. In some embodiments, the ovaries are stimulated, for example to allow maturation of more than one egg per month. In some embodiments, the ovaries are stimulated using gonadotrophin injections. Alternatively or additionally, the ovaries are stimulated by ovarian stimulation medications, for example derivatives of follicle-stimulating hormone (FSH) and/or luteinizing hormone (LH).

According to some exemplary embodiments, the maturation of the follicles is monitored at 104. In some embodiments, the maturation is monitored using ultrasound optionally for measuring the growth of each follicle and the thickness of the uterine lining. Additionally or optionally, the blood levels of hormones, for example estrogen are tested to monitor the maturation of the follicles.

According to some exemplary embodiments, final maturation is triggered at 106. In some embodiments, final maturation is triggered approximately 36 hours prior to Oocyte retrieval, for example 38 hours, 36 hours, 34 hours, 30 hours or any intermediate smaller or larger value.

According to some exemplary embodiments, an Oocyte is retrieved at 108. In some embodiments, the Oocyte is retrieved by insertion of a hollow needle into a follicle, optionally under ultrasound imaging. In some embodiments, the content of the follicle is removed, optionally by applying suction forces. In some embodiments, the follicle content comprises FF and an Oocyte.

According to some exemplary embodiments, the follicle content is stored at 110. In some embodiments, the follicle content of each follicle is stored separately from the content of other follicles. Optionally the FF is stored together with the Oocyte. Alternatively, the FF is stored separately from the Oocyte. In some embodiments, once the content of one follicle is isolated and stored, a new Oocyte is retrieved at 108. Optionally, each FF storage compartment and each respective Oocyte containing storage compartment are labeled, for example to allow matching of a stored FF sample to a stored Oocyte.

According to some exemplary embodiments, the stored Oocyte is cryopreserved at 112. In some embodiments, the stored Oocyte is optionally washed and placed in freezing media prior to cryopreservation.

According to some exemplary embodiments, the stored FF is analyzed at 114. In some embodiments, the FF is analyzed to determine at least one parameter related to the state of the respective stored Oocyte, for example a vitality level of the Oocyte, maturation level of the Oocyte, cleavage rates, Oxidative level of the Oocyte, the ability of the Oocyte, the ability of the Oocyte to remain viable post freezing and/or thawing or any other parameter related to the success of an IVF procedure and/or a cryopreservation procedure using the selected Oocyte. Alternatively or additionally, the FF is analyzed to determine the genetic profile of the Oocyte. In some embodiments, based on the analysis results a specific Oocyte will be selected and thawed, for example to undergo IVF.

According to some exemplary embodiments, a selected Oocyte undergoes IVF at 116. In some embodiments, the Oocyte is selected based on the analysis results at 114. In some embodiments, the selected Oocyte is placed in a plate together with sperm cells at 116. In some embodiments, the sperm cells are added to the plate media. Alternatively, a single sperm cell is injected by an expert into the Oocyte.

According to some exemplary embodiments, the embryo development is monitored at 118. In some embodiments, the embryo development is monitored until the formation of 6 to 10 cell embryos, which can optionally be transferred back to the uterus as 2-3 day-embryos. Alternatively, the embryo development is monitored until the formation of a blastocyst, which can optionally be transferred back to the uterus as 5-day embryo. In some embodiments, and without being bound to any theory, day 5 transfers may be ideal since in natural conception cycles, embryos typically implant on days 5 or 6 after ovulation. According to this theory, day 5 or 6 blastocyst embryo transfers may be preferable due to the ideal uterine environment conditions at this stage.

In some embodiments, the embryo development is monitored, for example to determine which of the developing embryos to transfer to the Uterus at 120. Alternatively or additionally, the embryo development is monitored, for example to determine which of the developing embryos to cryopreserve at 122. Optionally, the decision which of the embryos to transfer at 120 to the Uterus for further development is based on the results of the FF analysis at 114. For example, only embryos that have a high probability for normal development, as determined based on the FF analysis at 114, will be transferred to the Uterus at 120. Optionally, the decision which of the embryos to cryopreserve is based on the results of the FF analysis at 114. For example, only embryos that have a high probability to be properly thawed and/or to survive long term cryopreservation based on the FF analysis at 114, will be cryopreserved.

### Exemplary Process for Follicular Content Isolation, Separation and Storage

Reference is now made to FIG. 1B depicting a process for follicular content isolation, separation and storage, according to some exemplary embodiments of the invention.

According to some exemplary embodiments, at least part of a hollow probe, for example at least part of a hollow needle, optionally the distal end of the probe is introduced into an ovary at 132. Optionally, the probe is introduced into the ovary under ultrasound imaging. Alternatively or additionally, the probe is introduced into the ovary under the inspection of an optical assembly, for example a camera or an optic sensor. In some embodiments, the probe is introduced into the ovary during Oocyte retrieval 108.

According to some exemplary embodiments, a follicle is selected at 134. In some embodiments, the follicle is selected based on the size and/or the shape of the follicle. Optionally, the size and/or the shape are determined using the ultrasound imaging. Alternatively or additionally, the size and/or the shape are determined using the optical assembly.

According to some exemplary embodiments, at least part of the hollow probe is inserted into the selected follicle at 136. Optionally, the distal end of the hollow probe is inserted into the selected follicle at 136. In some embodiments, the hollow probe is inserted into the selected follicle under ultrasound imaging or under visualization using an optical assembly.

According to some exemplary embodiments, the content of the follicle is removed at 138. In some embodiments, the content of the follicle comprises an Oocyte and FF. Optionally, the content of the follicle is removed by application for suction forces, for example generating vacuum at a lumen of the probe. In some embodiments, the lumen of the probe is part of a flow path, for example a follicle content isolation flow path. In some embodiments, the content of the follicle is removed under ultrasound imaging and/or other visualization means, for example to make sure an Oocyte is isolated and/or to make sure that the follicle is empty post isolation and before a new follicle is selected.

According to some exemplary embodiments, the isolated follicular content is stored at 140. In some embodiments, the follicular content is stored in a storage compartment connected to an opening of the follicle content isolation flow path, for example a proximal opening positioned outside the body. In some embodiments, the Oocyte and the FF from the same follicle are stored together in the same storage compartment.

According to some exemplary embodiments, the Oocyte and the FF isolated from each follicle are separated at 142. In some embodiments, the follicular content passes through a separator, for example a filter found in the isolation flow path. Optionally, the filter comprises pores in the filter wall sized and shaped to allow only FF to pass through the pores into a different flow path, for example a FF isolation flow path.

According to some exemplary embodiments, the Oocyte is stored at 144. In some embodiments, the Oocyte flows within the isolation flow path to a storage compartment, for example an Oocyte storage compartment connected to a proximal opening of the isolation flow path located outside the body. In some embodiments, the Oocyte storage compartment comprises a tube, for example a centrifugation tube or a cuvette configured to be placed inside an optical analyzer. Optionally, the Oocyte passes through at least one additional separator, for example a filter, shaped and sized to allow removal of residual tissue, for example parts of membranes, cell debris prior to Oocyte storage. In some embodiments, removal of cell debris and residual tissue parts, for example membranes is important to reduce contamination of the stored follicle. Optionally, the Oocyte is transferred to an Oocyte storage compartment already filled with a solution, for example a buffer, a washing solution or buffer, a storing solution or buffer, a freezing solution or buffer or any type of solution.

According to some exemplary embodiments, the FF is stored at 146. In some embodiments, the FF passing through the pores of the filter is collected by a FF storage compartment, for example at vial or a tube. Optionally, the FF storage compartment comprises a testing tube or a testing cuvette. In some embodiments, the testing tube or testing cuvette is configured to be placed in a centrifuge or to be placed in an optical analyzer. In some embodiments, the FF storage compartment comprises a testing strip, optionally embedded in the storage compartment, for example for determining levels of at least one parameter of the FF, as described previously, for example nitric oxide (NO), malondialdehyde (MDA), reduced glutathione (GSH), or any other indicators of the oxidative status of the follicle, for example to predict the outcome of in vitro fertilization.

According to some exemplary embodiments, the follicle content is analyzed at 148. In some embodiments, the follicle content for example, the Oocyte and/or the FF is analyzed at 148. In some embodiments, the follicle content is analyzed optically, by an optical analyzer and/or an optical sensor and/or a microscope. Alternatively or additionally, the follicle content is analyzed chemically, for example by a testing strip. Alternatively or additionally, the follicle content is genetically analyzed, for example by a genetic sequencer, and/or a polymerase chain reaction (PCR) or any other genetic analyzer. In some embodiments, the stored FF is analyzed, for example as described herein, to determine the state or condition of an Oocyte isolated from the same follicle which is optionally stored separately from the FF.

According to some exemplary embodiments, once the content of a follicle is isolated, the storage compartment at the proximal end of the isolating flow path is replaced at 150. Optionally, the storage compartment is replaced when the needle penetrates into a new follicle or when changes in pressure in the isolation flow path are detected. Alternatively, the storage compartment is replaced when changes in electrical voltage or current of an electrical vacuum pump used to generate negative pressure within the isolation flow path are detected. Optionally, the Oocyte storage compartment and/or the FF storage compartment are replaced at 150. Optionally, the separator is replaced, for example if the pores are clogged with material that prevents flow of FF to the FF storage compartment.

According to some exemplary embodiments, the fluid flow path is cleaned at 152. In some embodiments, the fluid flow path, for example the isolation flow path is cleaned before the isolating the follicular content of a new follicle. Optionally, the isolation flow path is cleaned by washing the flow path with fluid, for example a washing solution, saline, phosphate-buffered saline or any other solution. Alternatively or additionally, vacuum is applied or air is pushed into the flow path, for example for cleaning the flow path and/or for drying the residual washing solution. In some embodiments, the FF isolation flow path and/or the Oocyte isolation flow path are cleaned in a similar way.

According to some exemplary embodiments, a new follicle is selected at 134 optionally under ultrasound imaging.

### Exemplary System for Follicular Content Isolation, Separation and Storage

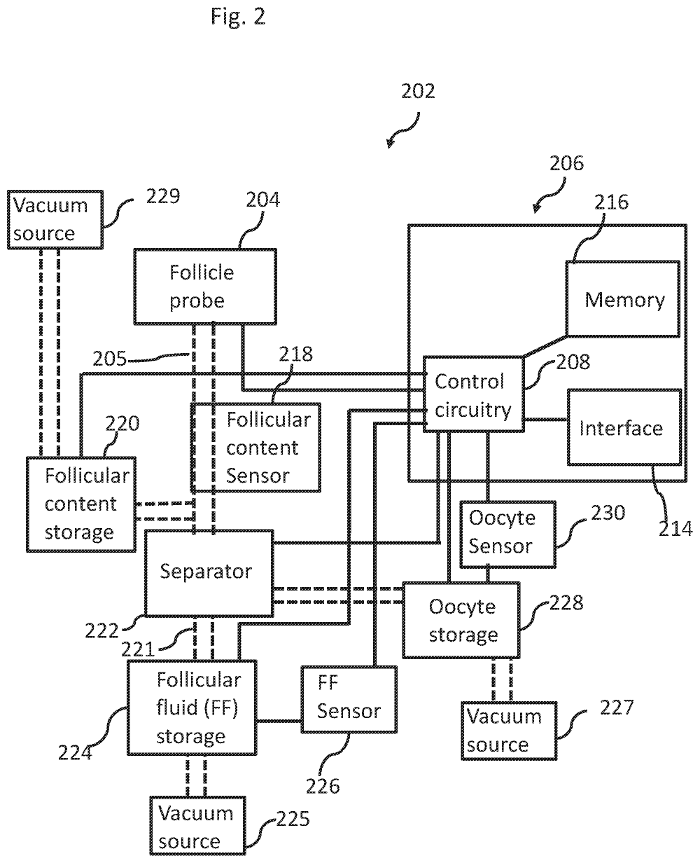

According to some exemplary embodiments, a system for isolation, separation and storage of follicular content is configured to remove the content of a plurality of follicles, where the content of each follicle is stored separately from the content of the rest of the follicles. Reference is now made to FIG. 2, depicting a system for isolation, separation, storage and analysis of follicular content, according to some exemplary embodiments of the invention.

According to some exemplary embodiments, a system, for example system 202 for isolation and storage of follicular content comprises a control unit, for example a control unit 206, a follicle probe 204, for example a hollow needle and at least one storage compartment, for storing the follicular content. Alternatively or additionally, the at least one storage compartment is configured to store an Oocyte from each follicle separately from the rest of the Oocytes. Optionally, the at least one storage compartment is configured to store the FF of each follicle separately from the FF of other follicles.

According to some exemplary embodiments, the follicle probe, for example a needle is shaped and sized to be insertable into a follicle optionally a human female follicle. In some embodiments, the needle is an elongated hollow needle having a distal opening shaped and sized to be insertable into the follicle and a proximal opening configured to be connected to a flow path, for example an isolation flow path 205. Optionally, the distal opening is positioned at a distal tip of the needle.

According to some exemplary embodiments, the follicle probe, for example the needle comprises at least one sensor, for example pressure sensor, a position sensor or a needle orientation sensor. In some embodiments, the pressure sensor is configured to measure the pressure and/or changes in pressure within a lumen of the hollow needle. Alternatively, the pressure sensor is configured to measure pressure and/or changes in pressure within the follicle. In some embodiments, the position sensor is configured to determine a relative position of the needle and/or the needle tip, for example to determine whether the needle tip is within a follicle or positioned outside the follicle. In some embodiments, the needle orientation sensor is configured to sense the orientation of the needle and/or changes in the needle orientation.

According to some exemplary embodiments, a fluid flow, for example fluid flow 205 is connected to the proximal opening of the follicle probe 204. In some embodiments, fluid flow path 205 is a tube optionally a flexible tube made from silicone or rubber. In some embodiments, fluid flow path 205 comprises at least one proximal opening positioned outside the body, connected to a follicular content storage compartment 220. Alternatively or additionally, the fluid flow path comprises at least one opening connected to a follicular fluid (FF) storage compartment 224 and/or to an Oocyte storage compartment 228.

According to some exemplary embodiments, a separator 222, for example a filter is positioned on the flow path 222. In some embodiments, an internal lumen of the separator is aligned with flow path, for example to allow fluid flow in the fluid flow path through the separator. In some embodiments, the separator comprises a plurality of pores in at least part of the separator wall in a size range of 1 micron (μm) to 50 μm, for example 1 μm, 5 μm, 7 μm, 10 μm or any intermediate smaller or larger pore size. In some embodiments, the pore size is selected to allow passing of FF through the pores into a follicular fluid flow path 221, and to prevent the passage of an Oocyte to the follicular fluid flow path 221.

According to some exemplary embodiments, the follicular content is removed from the follicle into the follicular content storage 220 when a vacuum source, for example vacuum source 229 connected to the follicular content storage 220.

Alternatively or additionally, an Oocyte is removed into the Oocyte storage 228 when a vacuum source, for example vacuum source 227 connected to the Oocyte storage 228 is activated. In some embodiments, vacuum source 227, vacuum source 225 and/or vacuum source 229 comprise a vacuum pump.

According to some exemplary embodiments, the FF passes through the pores, for example when a vacuum source, for example vacuum source 225 connected to the follicular fluid storage is activated, and optionally generates negative pressure within the follicular fluid flow path 221.

According to some exemplary embodiments, at least one sensor is positioned on the isolation flow path 205, for example follicular content sensor 218. In some embodiments, sensor 218 comprises a pressure sensor configured to sense pressure levels and/or changes in pressure levels within the isolation fluid flow path, for example when follicular fluid is removed from the follicle and the follicle gradually collapses. Alternatively or additionally, the sensor 218 comprises a flow sensor, configured to sense fluid flow or changes in fluid flow within the isolation fluid flow path, for example during the removal of follicular content from the follicle.

According to some exemplary embodiments, the sensor 218 comprises an optical sensor, configured to sense changes in at least one optical parameter or to measure values of the optical parameter during the isolation of follicular content. In some embodiments, the optical sensor is configured to sense when an Oocyte passes within the isolation flow path, for example based on at least one optical parameter of the Oocyte. In some embodiments, the optical parameter comprises light absorbance, light transmission and/or light diffraction.

According to some exemplary embodiments, the system 202 comprises at least one sensor, for example sensor FF sensor 226 and/or oocyte sensor 230 configured to measure at least one parameter of the content of the storage compartment. In some embodiments, FF sensor 226 is configured to measure the quantity of the FF within the FF storage compartment 224. Additionally or alternatively, the oocyte sensor is configured to detect an oocyte in the oocyte storage compartment 228.

According to some exemplary embodiments, system 202 comprises a control unit, for example control unit 206. In some embodiments, the control unit 206 comprises a control circuitry 208 electrically connected to one or more of follicle probe 204 and/or separator 222. Optionally, the control circuitry 208 is electrically connected to at least one vacuum source, for example vacuum source 229, vacuum source 225 or vacuum source 227, positioned on the isolation flow path from the follicle probe to one or more of the storage compartment. Additionally, the control circuitry 208 is electrically connected to at least one sensor, for example follicular content sensor 216, FF sensor 226, or oocyte sensor 230.

According to some exemplary embodiments, the control unit 206 comprises an interface 214 electrically connected to the control circuitry 208, configured to receive input from a user of the system 202 and/or to deliver a human detectable indication to the user. In some embodiments, the interface 214 comprises at least one light emitter configured to deliver a human detectable light indication to the user. Alternatively or additionally, the interface 214 comprises at least one sound generator, configured to generate and deliver a sound indication to the user.

According to some exemplary embodiments, the control circuitry 208 signals the interface 214 to generate the human detectable indication in response to electrical signals received from the follicle probe 204 and/or in response to electrical signals received from the separator 222. Alternatively or additionally, the control circuitry 208 signals the interface 214 to generate the human detectable indication in response to electrical signals received from at least one vacuum source and/or in response to electrical signals received from at least one sensor, for example follicular content sensor 216, FF sensor 226, or oocyte sensor 230.

According to some exemplary embodiments, the control unit 208 comprises a memory 216 electrically connected to the control circuitry 208. In some embodiments, memory stores log files of the system. Alternatively or additionally, the memory 216 stores values or indications of values of at least one parameter related to the isolation of follicular content from the follicle, for example pressure levels, amount of isolated material or any other related parameter values.

### Exemplary Process for Monitoring Follicular Content Isolation

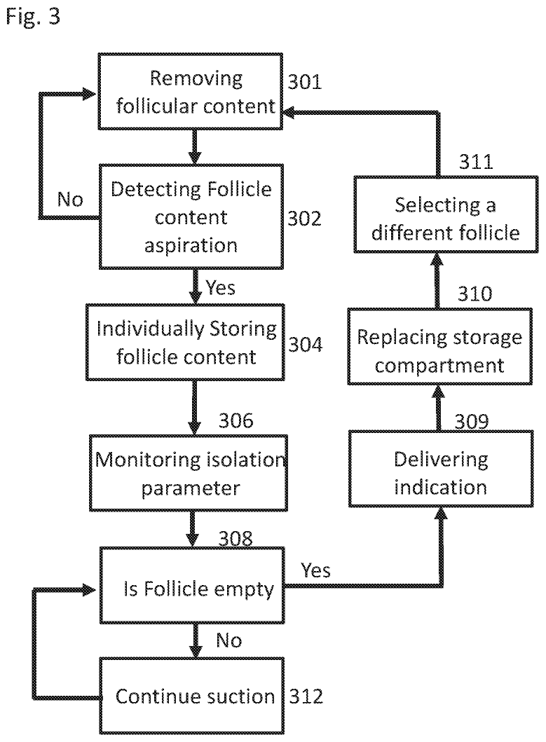

According to some exemplary embodiments, a follicular content isolation process is monitored, optionally by the control unit 206 shown in FIG. 2, for example to determine when a follicle is empty and/or when an Oocyte is isolated. Reference is now made o FIG. 3 depicting a process for monitoring a follicular content isolation process, according to some exemplary embodiments of the invention.

According to some exemplary embodiments, follicular content is removed at 301. In some embodiments, the follicular content is removed by penetrating with at least part of a follicle probe, for example a needle into a follicle, optionally under ultrasound imaging. In some embodiments, when at least part of the needle, optionally a distal part of the needle is positioned within the follicle a signal is delivered to the control circuitry 208 by at least one sensor, for example a needle position sensor and/or a needle orientation sensor. Alternatively or additionally, a user delivers a signal to the control circuitry 208 through interface 214. In some embodiments, the control circuitry activates a vacuum source on an isolation flow path, for example flow path 205 in response to the received signals.

According to some exemplary embodiments, follicle content aspiration is detected at 302. Optionally, the follicle content aspiration is detected by at least one sensor, for example an optical sensor configured to sense flow inside the isolation flow path and/or inside the needle. Alternatively or additionally, the follicle content aspiration is detected by at least one flow sensor configured to sense flow within the isolation flow path and/or inside the needle. Optionally, an indication is delivered to a user, for example by interface 214 when aspiration is detected. Alternatively, an indication, for example an alert signal is delivered to a user optionally by interface 214 when aspiration is not detected. In some embodiments, is aspiration is not detected then the needle is repositioned within the Ovary, for example by selecting a different oocyte.

According to some exemplary embodiments, the follicular content is separately stored in a storage compartment at 304. In some embodiments, the follicular content, for example FF and Oocyte is stored in a storage compartment connected to the isolation flow path.

According to some exemplary embodiments, at least one follicular content isolation parameter is monitored at 306. In some embodiments, the isolation parameter comprises one or more of flow and/or flow changes within the isolation flow path, flow and/or flow changes within the needle, pressure and/or changes in pressure within the isolation flow path, pressure and/or changes in pressure within the needle. Alternatively or additionally, the isolation parameter comprises the amount of isolated follicular content. Optionally, the isolation parameter is measured by signals transmitted to the control circuitry 208 from at least one sensor positioned in the needle and/or at least one sensor positioned on the isolation flow path and/or at least one sensor positioned within the isolation flow path. In some embodiments, if the measured isolation parameter values are higher or lower than pre-determined values and/or are not in a pre-determined range of values and indication is delivered, optionally by the interface 214.

According to some exemplary embodiments, the system or a user of the system determines if the follicle is empty at 308 optionally based on the measured parameters values at 306. In some embodiments, the system or the user determine if the follicle is empty based on pressure levels and/or changes in pressure levels within the isolation flow path. Alternatively or additionally, the system or the user determine if the follicle is empty based on fluid flow levels and/or changes in fluid flow levels within the isolation fluid flow path. In some embodiments the follicle is empty, when there is a reduction in fluid flow and/or when fluid flow stops. In some embodiments, the system or the user determine if the follicle is empty based on voltage and/or amperage changes of a vacuum source, for example one or more of vacuum sources 229, 225 or 227, used to generate a negative pressure inside the isolation flow path.

According to some exemplary embodiments, if the follicle is not empty then suction continues.

According to some exemplary embodiments, if the follicle is empty then an indication is delivered at 309. Optionally, the indication is delivered by the interface 214 and comprises a human detectable indication, for example a light indication and/or a sound indication.

According to some exemplary embodiments, a storage compartment for follicular content is replaced at 310. In some embodiments, the storage compartment is replaced when the follicle is empty or when a desired amount of material was collected. Alternatively or additionally, the storage compartment is replaced when an Oocyte is detected in the storage compartment, optionally by a sensor configured to monitor at least one parameter related to the content of the storage compartment, for example amount of FF and/or the presence of the Oocyte. In some embodiments, the storage compartment is replaced automatically by the system. Alternatively, a user manually replaces the storage compartment, for example following an indication received from the system.

According to some exemplary embodiments, a new follicle is selected for follicular content isolation at 311. In some embodiments, a user selects a new follicle when a previous follicle is empty and/or when an oocyte is removed from the previous follicle.

### Exemplary Needle

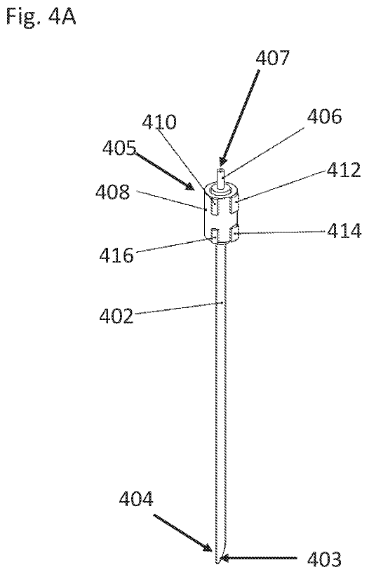

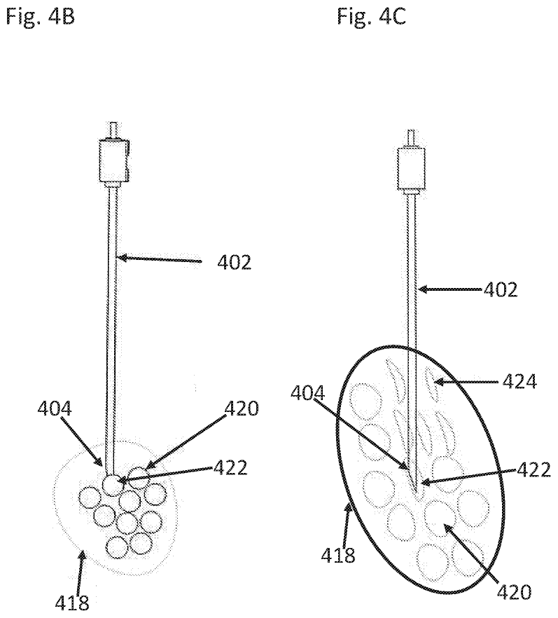

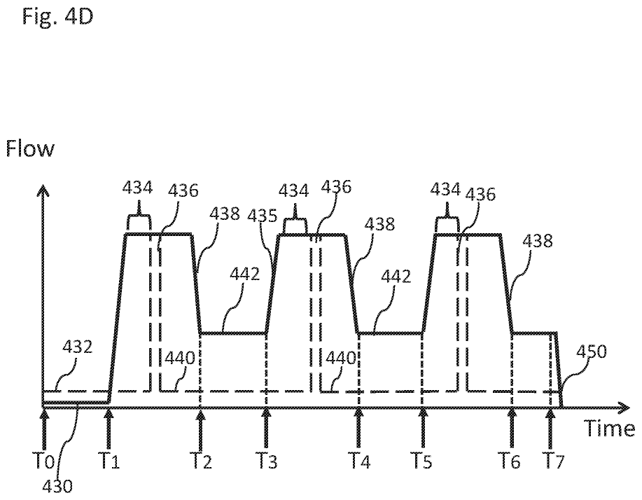

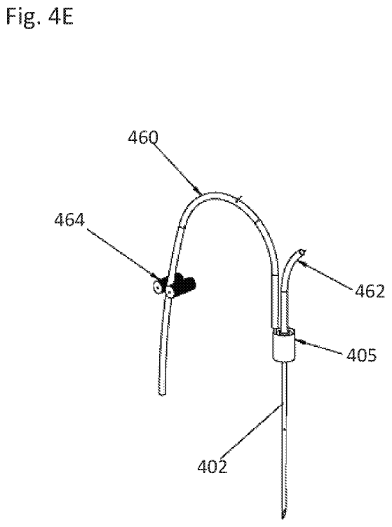

According to some exemplary embodiments, a follicle probe comprises an elongated hollow needle which is shaped and sized to be insertable into a follicle, and to isolate the follicle content through a needle lumen. Additionally, the needle comprises at least one opening connectable to a fluid flow path defined from the needle and into a storage compartment. Reference is now made to FIG. 4A depicting a needle shaped and sized for penetration into a follicle and removal of follicular content, according to some exemplary embodiments of the invention.

According to some exemplary embodiments, an elongated hollow needle 402 comprises a distal end 404 optionally a tapered distal end shaped and sized to penetrate through follicle membranes and a proximal end 407. In some embodiments, the needle 402 comprises a distal opening 403 near or at the distal end 404. In some embodiments, the needle 402 has an outer diameter in a range of 0.5-1.7 mm, for example 0.9081 mm, 1.067 mm, 1.27 mm, 1.473 mm, 1.651 mm or any intermediate, smaller or larger diameter value. In some embodiments, the needle 402 has an inner diameter in a range of 0.5-1.7 mm, for example 0.686 mm, 0.838 mm, 1.067 mm, 1.194 mm or any intermediate, smaller or larger value. Optionally, needle 402 is a needle with a Birmingham gauge is a range of 16G-19G. In some embodiments, the diameter of the distal opening is large enough to allow penetration of follicular fluid and an Oocytes through the opening into a lumen on the needle 402.

According to some exemplary embodiments, a tubing 406 is optionally positioned within the lumen of the needle 402. In some embodiments, the tubing 406 comprises a proximal opening 407 connectable to a fluid flow path, for example fluid flow path 205, shown in FIG. 2. Alternatively, the fluid flow path is connectable to a proximal opening of the needle 402.

According to some exemplary embodiments, the needle 402 comprises a flexible circuit board, for example a flexible printed circuit board (PCB) 408. In some embodiments, the needle 402 comprises at least one sensor, for example a position sensor and/or an orientation sensor, for example tilt sensor 416 configured to sense the orientation of the needle or the orientation of the distal end of the needle. Optionally the tilt sensor 416 is an accelerometer. In some embodiments, the at least one sensor comprises a position sensor configured to sense the position of the needle within the ovary or within the follicle. In some embodiments, the at least one sensor is part of the PCB 408.

According to some exemplary embodiments, the PCB 408 comprises at least one pressure sensor or at least one pressure or vacuum gauge, for example vacuum gauge 410 to measure or sense pressure or vacuum levels and/or changes in pressure or vacuum levels within the needle lumen.

According to some exemplary embodiments, the PCB 408 comprises at least one control circuitry, for example needle control circuitry 414 and optionally a power source, for example a battery 412 electrically connected to the control circuitry 414. In some embodiments, PCB 408 comprises a battery sensor electrically connected to the battery 412 and to the control circuitry 414 for transmitting a signal to the control circuitry when the electric power in the battery is below a pre-determined value or to continuously monitor the power level in the battery 412.

According to some exemplary embodiments, the needle control circuitry 414 is electrically connected to the control circuitry of the system, for example control circuitry 208 shown in FIG. 2. Optionally, the needle control circuitry 414 is wirelessly connected, for example by Bluetooth, Wi-Fi or any wireless signal to a receiver in the control unit, for example control unit 206. Alternatively or additionally, the needle control circuitry 414 is wirelessly connected, for example by Bluetooth, Wi-Fi or any wireless signal to the control circuitry 208.

According to some exemplary embodiments, the control circuitry 208 receives information on the movement of the needle based on signals received from at least one sensor of the needle, for example a tilt sensor or a position sensor. Additionally or alternatively, the control circuitry determines if a follicle is empty based on signals received from the pressure or vacuum gauge 410 of the needle. In some embodiments, based on signals received from the pressure or vacuum sensor, the control circuitry 208 delivers a signal to replace a storage compartment.

Exemplary Needle Penetration into Follicle

According to some exemplary embodiments, a follicle probe, for example a needle is guided into a follicle within the ovary, optionally under ultrasound imaging, to remove the follicle content. In some embodiments, a system for example, system 202 shown in FIG. 2, senses when the follicle is empty, and provides an indication to a user. In some embodiments, the system senses when the follicle is empty based on signals received from at least one sensor positioned on a flow path between the needle and a storage compartment, and/or based on at least one sensor positioned on the needle, for example a s shown in FIG. 4A. Reference is now made to FIGS. 4B and 4C depicting the penetration of a needle into a selected follicle, according to some exemplary embodiments of the invention.

According to some exemplary embodiments, a hollow follicle probe, for example needle 402 penetrates through an ovary membrane 418 and placed adjacent to a follicle 422. In some embodiments, for example as shown in FIG. 4C a distal end 404 of the needle penetrates through the follicle 422 membrane. Optionally, a tapered section at the distal end 404 of the needle penetrates into the follicle, positioning a distal opening of the needle inside the follicle.

According to some exemplary embodiments, during the removal of follicular content, the follicle 422 collapses on the distal end 404 of the needle. In some embodiments, the collapse of the follicle on the distal end of the needle causes at least a partial closure of a distal opening of the needle. Optionally, the at least partial closure of the distal opening causes disruption of fluid flow through the needle and/or disruption of pressure values within the needle lumen. In some embodiments, at least one sensor, for example a pressure sensor or a flow sensor senses these disruptions and delivers an electric signal to a control circuitry of the system, for example control circuitry 208.

Reference is now made to FIG. 4D of a graph depicting changes in flow during the isolation of follicular content from a plurality of follicles, according to some exemplary embodiments of the invention.

According to some exemplary embodiments, when a needle penetrates into a follicle, vacuum forces are applied at T1 and flow of material from the follicle, for example flow of follicular content, within the isolation flow path increases, as shown by graph 430. In some embodiments, when a predetermined amount 434 of follicular content passes through the isolation flow path, a selected amount of follicular fluid is diverted into a follicular fluid flow path, for example as shown by graph 432 which represents flow in the follicular fluid flow path. In some embodiments, flow in the follicular fluid flow path increases for a short time period, for example to collect a pre-determined amount of follicular fluid from a specific follicle. In some embodiments, the pre-determined amount of follicular fluid is at least 10 microliter (μl), for example 20 μl, 50 μl, 100 μl, 200 μl, 500 μl or any intermediate, smaller or larger amount of follicular fluid.

According to some exemplary embodiments, the follicular fluid passes into the follicular fluid flow path, when a vacuum source connected to the follicular fluid flow path is activated, and optionally generates a negative pressure for a selected time period within the follicular fluid flow path. Optionally, a separator, for example a filter positioned at the connection between the fluid isolation flow path and the follicular fluid flow path prevents the entry of oocytes into the follicular fluid flow path. In some embodiments, when the predetermined amount of FF passes through the FF flow path, active flow is stopped and the flow levels in the FF flow path return to baseline levels of passive flow 440. Optionally, the active flow is stopped by deactivating the vacuum source of the FF flow path.

According to some exemplary embodiments, when the follicle is empty, fluid flow in the follicular content isolation flow path decreases as shown by negative slope of 438. Optionally, when the needle passes through the follicle into a void between follicles at T2, flow within the isolation flow path is reduced to baseline levels 442. In some embodiments, when the needle penetrates into a new follicle at T3, flow increases, as shown by the positive slope 435.

According to some exemplary embodiments, the process of isolation follicular content from a specific follicle and diverting the FF from each follicle through the FF flow path is repeated in a plurality of follicles. In some embodiments, the needle exits through the last follicle at T6 and the isolation process is stopped at T7, where the flow levels in the isolation flow path are reduced to zero, as shown at 450.

According to some exemplary embodiments, a control circuitry of the system, for example control circuitry 206, measures flow levels within the isolation flow path and determines based on the measured flow levels when to initiate active flow in the FF flow path, for example to collect FF that optionally be used to determine a state of an Oocyte isolated from the same follicle. Additionally, the control circuitry 206 measures flow levels and/or changes in flow levels with the isolation flow path, for example to determine when a follicle is empty. Optionally, the control circuitry 206 determines that follicle is empty when flow in the isolation flow path is reduced, for example as demonstrated by slope 438.

### Exemplary Tube Flushing

According to some exemplary embodiments, the oocyte from each follicle is isolated and stored separately from other stored oocytes. Additionally, FF is collected during the isolation of follicular content from each follicle, for example to determine at least one parameter related to the condition or state of the oocyte taken from the same follicle. In some embodiments, to avoid cross contamination between the FF isolated from one follicle with FF isolated from a second follicle, the isolation flow path and/or the FF flow path is optionally flushed. In some embodiments, the allowed cross contamination of an isolated FF with FF from a different follicle is up to 10% of the volume, for example 10%, 8%, 5% or any intermediate, smaller or larger percentage of cross contamination. Reference is now made to FIG. 4E depicting flushing and/or squeezing out residual material from the isolation flow path, according to some exemplary embodiments of the invention.

According to some exemplary embodiments, needle 402 comprises a needle head 405 with two openings to the needle lumen. In some embodiments, one of the openings is connectable to an isolation fluid flow path, for example suction tube 460. In some embodiments, a second opening is connectable to a flushing fluid flow path, for example flush tube 462. Optionally, the flushing fluid flow path is connected a flushing fluid source. In some embodiments, flushing fluid is delivered through the flushing tube into the needle, for example to flush residual follicular material from the lumen of the needle. Alternatively or additionally, the flushing fluid is delivered through the isolation flow path, for example suction tube 460, for example to remove residual follicular content material from the suction tube 460. Optionally, a flushing fluid source is connected via a flushing tube to the FF flow path. In some embodiments, the flushing fluid is delivered through the FF flow path, for example to prevent contamination of FF isolated from one follicle with FF isolated from a different follicle. In some embodiments, the flushing fluid comprises a saline solution, a phosphate-buffered saline (PBS) solution or any other bio compatible solution.

According to some exemplary embodiments, during a flushing cycle, the flushing fluid is delivered through the isolation flow path and/or through the FF flow path, for example when the needle passes from one follicle to another follicle or when the follicle is empty. In some embodiments, the flushing cycle is activated optionally automatically, when the follicle is empty or prior to the penetration of the needle into a new follicle.

According to some exemplary embodiments, when a flushing cycle is activated, the isolation fluid flow path and/or the FF flow path and/or the oocyte flow path are connected to waste storage compartments. Alternatively, during flushing, part of the isolation flow path is blocked to allow flow of flushing fluid only into the FF flow path.

According to some exemplary embodiments, a squeezer, for example squeezer 464 is positioned on the follicular content isolation flow path, for example suction tube 460. In some embodiments, the squeezer is configured to press the suction tube from at least two opposite sides, for example to squeeze out any residual material left inside the suction tube. Optionally, the squeezer is movable and configured to move along the suction tube while pressing on the tube walls.

Exemplary Process for Separation of Follicular Fluid from Oocyte

According to some exemplary embodiments, during an egg retrieval process where the follicular content from a plurality of follicles is removed, the content of each follicle is isolated and stored separately from the content of other follicles. In some embodiments, during the removal of follicular content from each follicle, at least some FF isolated from the follicle is stored separately from the oocyte. In some embodiments, the FF is analyzed to determine at least one parameter related to the state or condition of the oocyte that was isolated from the same follicle as the FF.

According to some exemplary embodiments, a database is generated which includes data on all the oocytes that are isolated in a single oocyte retrieval process from the same subject. In some embodiments, the database includes the analysis results of the FF, associated with a single specific oocyte that was isolated from the same follicle as the analyzed FF.

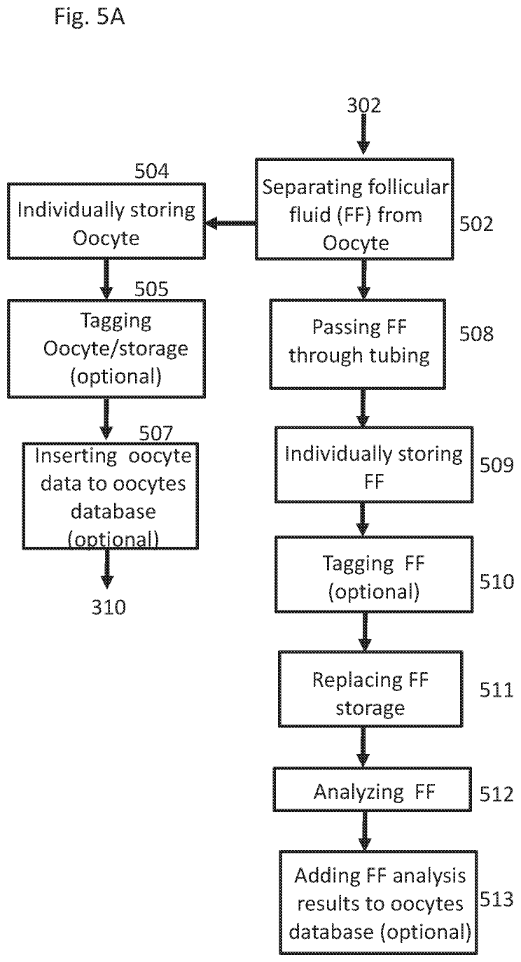

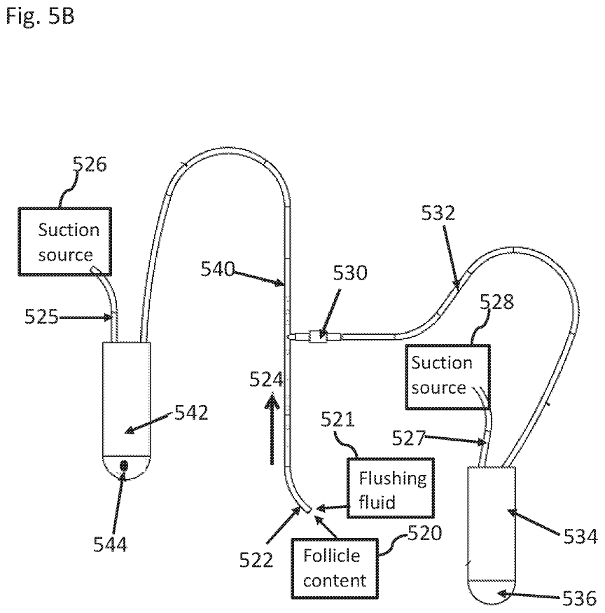

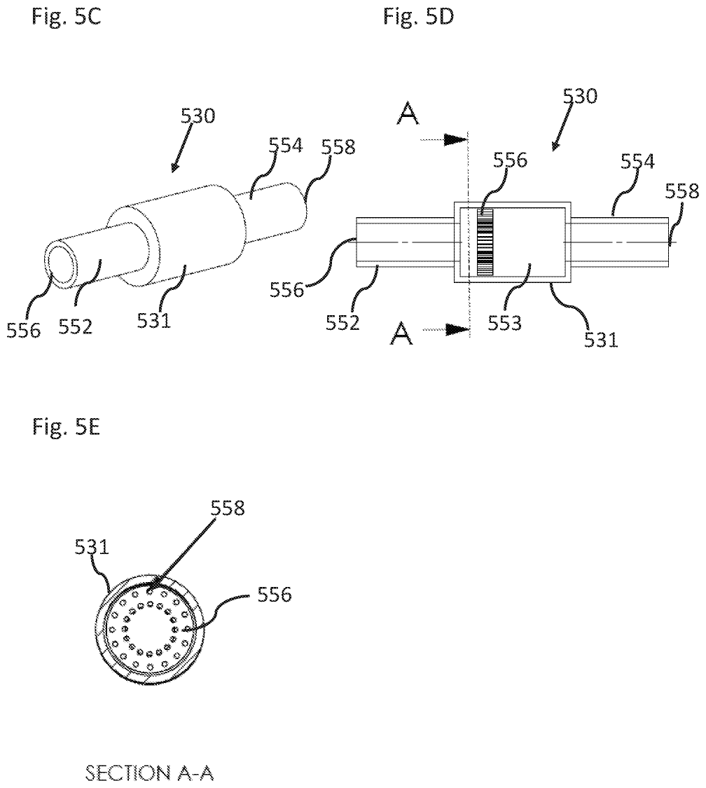

Reference is now made to FIG. 5A, depicting a flow chart of a process for separating a sample of FF from follicular content which comprises an oocyte, according to some exemplary embodiments of the invention.

According to some exemplary embodiments, once a follicle content aspiration is detected at 302, a system monitors the follicle content flow through an isolation flow path. In some embodiments, a FF sample is collected after the flow of a pre-determined amount of follicular content. In some embodiments, the FF sample is collected, for example after at least 500 microlitre (ul) are drawn from the follicle, for example after 1 ml, after 1.5 ml, after 2 ml or any intermediate, smaller or larger value. Optionally, the FF sample is drawn through a filter with pore sizes or the largest dimension of the pores are in a range of 2-50 micron, for example 3 micron, 5 microns, 10 microns, 20 microns, or any intermediate, smaller or larger size. In some embodiments, the pores size prevents the passage of Oocytes.

According to some exemplary embodiments, the FF is drawn through the tubing at 508. In some embodiments, the FF is drawn through a FF flow path towards a storage compartment, for example a FF storage compartment.

According to some exemplary embodiments, the FF is individually stored in the storage compartment at 509. In some embodiments, the FF is separately stored from the oocyte and the FF collected from other follicles.

According to some exemplary embodiments, the FF is tagged at 510. In some embodiments, the FF storage is tagged. Optionally the tag comprises an identification number or symbol or a code which allows matching of specific FF and/or a specific FF storage compartment to a specific oocyte or to a specific oocyte storage compartment.

According to some exemplary embodiments, the FF storage is replaced at 511. In some embodiments, the FF storage is replaced to a new FF storage compartment, when a needle penetrates into a new follicle. Optionally, the FF storage compartment is replaced for example to prevent contamination of FF from a first follicle with FF from a different follicle. In some embodiments, the allowed contamination of isolated FF with FF from a different follicle is up to 10% of the volume, for example 10%, 8%, 5% or any intermediate, smaller or larger percentage of contamination.

According to some exemplary embodiments, the FF is analyzed at 512. In some embodiments, the FF is analyzed, for example to determine the oxidative levels of the FF. Alternatively or additionally, the FF is analyzed using biological, chemical and/or genetical methods, for example to determine values of at least one parameter related to a condition of an oocyte, isolated from the same follicle as the FF.

According to some exemplary embodiments, the FF analysis results are added to an oocyte database. In some embodiments, the oocyte database includes information regarding individual oocytes isolated and stored separately from each other in the same oocyte retrieval process. In some embodiments, the information comprises oocyte-specific data derived from the FF analysis results.

According to some exemplary embodiments, while collecting a FF sample at 502, the oocyte flows in a follicular content isolation flow path or in an oocyte flow path towards an oocyte storage compartment at 504. In some embodiments, an oocyte isolated from a follicle is isolated and stored separately from oocytes isolated from other follicles during the same oocyte retrieval procedure.

According to some exemplary embodiments, the stored oocyte is tagged at 505. Alternatively or additionally, the oocyte storage compartment is tagged. In some embodiments, the oocyte or the oocyte storage compartment is tagged, for example to allow matching between a stored oocyte and a stored FF sample collected from the same follicle as the oocyte at 502.

According to some exemplary embodiments, the oocyte data is inserted into the oocytes database at 507.

According to some exemplary embodiments, an oocyte, optionally stored separately from other oocytes, is scored based on the data stored in the database. In some embodiments, the oocyte is cored based on the data in the database derived from the FF analysis. In some embodiments, the database includes data on each oocyte entry which comprises an indication related to the FF analysis and/or any additional information regarding to the oocyte, for example visualization-related data, morphological data, genetic data of the FF or the oocyte, genetic data of the female subject from which the oocyte and FF were isolated or any other data.

According to some exemplary embodiments, the oocyte is selected for an in-vitro fertilization (IVF) process or for cryopreservation based on the scoring. In some embodiments, the database comprises FF analysis values or indications per at least one oocyte entry in the database.

### Exemplary Separation System

Reference is now made to FIG. 5B depicting a system for separation of FF from an oocyte, both isolated from the same follicle, according to some exemplary embodiments.

According to some exemplary embodiments, follicle content 520 is introduced into a distal opening 522 of a follicle fluid isolation flow path 540. In some embodiments, activation of a suction source 526, connected to a storage compartment, for example a follicle content storage compartment 542, generates a negative pressure within the isolation flow path 540. In some embodiments, the generated negative pressure attracts the follicle content in direction 524 into the isolation flow path. In some embodiments, activation of suction force 526, optionally connected to storage compartment 542 via tube 525, generates vacuum within the isolation flow path. In some embodiments, the generated vacuum attracts the follicle content 520 into the isolation flow path 540. Optionally, the distal opening 522 of the isolation flow path is connected to a follicle probe, for example a needle.

According to some exemplary embodiments, the follicle content which comprises an oocyte and FF flows through the isolation flow path in direction 524. In some embodiments, the FF and the oocyte, both isolated from the same follicle are stored in storage compartment 542.

According to some exemplary embodiments, a FF flow path 532 is fluidically connected to the isolation flow path 540, optionally through a separator, for example separator 530. In some embodiments, separator 530 comprises a filter having pores with a maximal largest dimension of 50 micrometer (μm, micron), for example 2 micron, 5 microns, 10 microns, 20 microns, 50 microns or any intermediate, smaller or larger value. In some embodiments, the filter prevents passage of follicle material larger than the maximal largest dimension into the FF flow path 532. Optionally, FF penetrates through the pores into the FF flow path 532. In some embodiments, the FF flow path is connected to a FF storage compartment 534.

According to some exemplary embodiments, during the activation of suction source 526, only passive flow of FF penetrates through the filter into the FF flow path 532. In some embodiments, activation of a suction source 528 which is connected via tube 527 to the FF storage compartment generates a negative pressure with the FF flow path 532. Optionally, generation of the negative pressure causes an active flow of FF through the filter pores into the FF flow path 532 and the FF storage compartment 534. In some embodiments, the negative pressure within the FF flow path 532 is generated for a pre-determined time or until a sufficient amount of FF is collected at the FF storage compartment 534. In some embodiments, a sufficient amount of FF 536 is an amount of at least 10 microliter, for example 10 microliter, 50 microliter, 100 microliter, 200 microliter, 500 microliter, 1000 microliter or any intermediate, smaller or larger amount of FF 536 collected in the FF storage compartment 534.

According to some exemplary embodiments, in order to prevent contamination of the stored FF with residual FF found in the FF flow path 532 or in the isolation flow path 540, flushing is applied by delivery of Flushing fluid 521 through the FF flow path 532 and optionally through the isolation flow path 540. In some embodiments, the allowed contamination level of stored FF with residual FF from a different follicle is up to 10% of the volume, for example 10%, 8%, 5% or any intermediate, smaller or larger percentage of contamination. In some embodiments, the suction source 528 is activated to draw the flushing fluid into the FF flow path 532. Alternatively, both suction source 526 and suction source 528 to draw flushing fluid into the isolation flow path 540 and into the FF flow path 532. In some embodiments, flushing fluid is applied as part of a flushing cycle, optionally initiated when the needle is navigated into a new follicle. Optionally, when the flushing cycle is initiated, the FF storage compartment is replaced with a waste storage compartment. Alternatively or additionally, when the flushing cycle is initiated, the oocyte storage compartment 542 is replaced with a waste storage compartment. In some embodiments, the flushing cycle is applied for at least 4 seconds, for example for 5 seconds, 10 seconds, 30 seconds, 1 minute or any intermediate, smaller or larger time period.

Reference is now made to FIGS. 5C-5E depicting a FF separator, according to some exemplary embodiments of the invention.

According to some exemplary embodiments, a separator 530 comprises a central portion 531 with two cylindrical extensions, for example proximal extension 552, which is close to the isolation flow path, and a distal extension 554 which is close to the FF flow path. In some embodiments of the invention, the separator 530 is a filter or comprises a filter. In some embodiments, an internal diameter of the separator 530 is in a range of 0.4-2.5 mm, for example 0.4 mm, 0.5 mm, 0.8 mm, 1 mm or any intermediate, smaller or larger diameter. In some embodiments, the isolation flow path is connectable to the proximal section 552 through proximal opening 556. Alternatively, an adaptor positioned on the isolation flow path, for example a T adaptor, is connectable to the proximal section 552 of the separator 531 through opening 556. In some embodiments, a FF flow path is connectable to the distal section 554 of the separator through opening 558. In some embodiments, an internal diameter of the FF flow path, and/or an internal diameter of the isolation flow path is in a range of 0.4-2.5 mm, for example 0.4 mm, 0.5 mm, 0.8 mm, 1 mm or any intermediate, smaller or larger diameter.

According to some exemplary embodiments, for example as shown in FIGS. 5D and 5E, the central portion 531 comprises an internal surface 556 which divides an internal lumen 553 of the central portion 531 into two separate sections, a proximal section close to the proximal opening and a distal section close to the distal opening. In some embodiments, the internal surface 556 comprises a plurality of pores 558, with a largest dimension with a maximal size of 50 microns, for example 5 microns, 10 microns, 50 microns. In some embodiments, only particles smaller than 50 microns can cross the internal surface through the pores from the proximal section to the distal section.

According to some exemplary embodiments, for example as shown in FIG. 5F, a separator 560 is positioned on an isolation flow path 562. In some embodiments, a FF flow path 564 is fluidically connected to the separator 560. In some embodiments, when follicle content 520 flows within the isolation flow path 562 through the separator 560, only particles smaller than 50 micron penetrate through pores in an internal surface of the separator into the FF flow path 564. Optionally, only FF penetrates through the pores in the separator and into the FF flow path. In some embodiments, oocytes and other particles larger than 50 micron continue to flow in the isolation flow path 562.

### Exemplary System for Separately Isolation and Follicle Content Storage

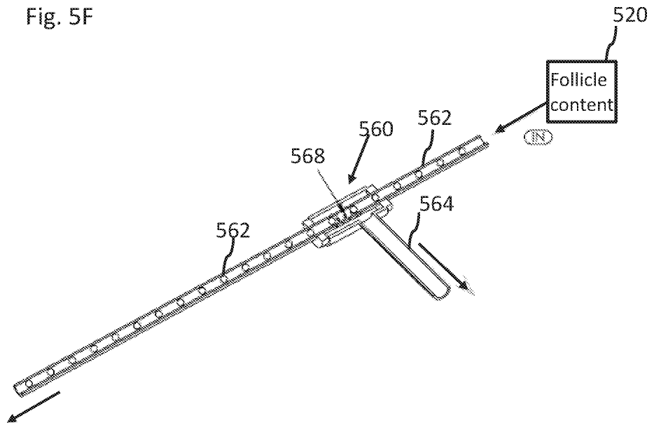

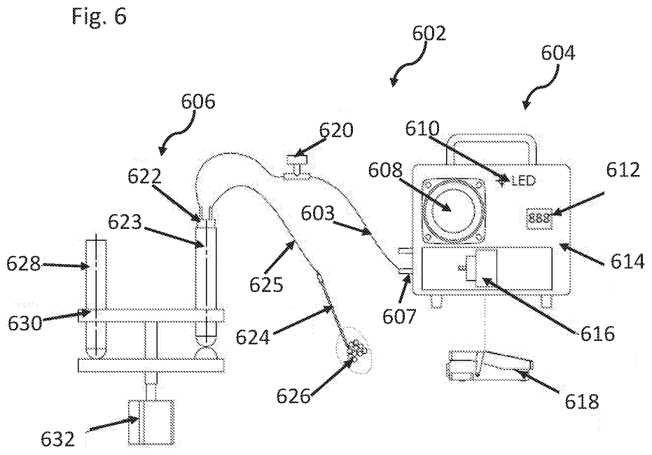

Reference is now made to FIG. 6 depicting a system for separately isolating and storing follicle content, according to some exemplary embodiments of the invention.

According to some exemplary embodiments, system 602 comprises a control unit 604 connectable to an isolation flow path, for example suction tube 603. In some embodiments, the suction tube 603 is connected to a sealing cover 622 of a storage compartment, for example storage compartment 623. In some embodiments, the isolation flow path is connected to a needle 624 by a tube 625. Optionally, the sealing cover 622 comprises a first connector and a second connector. In some embodiments, the first connector connects a suction tube 603 between the control unit and/or a vacuum source to the sealing cover 622. In some embodiments, the second connector connects the cover with tube 625 leading to the needle 624. Optionally, the cover comprises a sealing ring, optionally made from rubber in an interface between the cover and a storage compartment opening. Alternatively, the interface between the sealing cover 622 and the storage compartment opening is made from rubber.

According to some exemplary embodiments, the storage compartment 623 is placed in a cartridge 630 of a plurality of storage compartments, for example storage compartments 628 and 623. In some embodiments, the cartridge is a movable cartridge and comprises a motor which is configured to move the cartridge 630, for example to place a new storage compartment instead of a previous storage compartment.

According to some exemplary embodiments, the control unit 604 comprises a control circuitry connected to an interface configured to deliver indications to a user, for example sound indications and/or visual indications. In some embodiments, the interface comprises a speaker 608, and/or a screen 612 and/or a light-emitting indicator, for example LED indicator 610. In some embodiments, the interface comprises a user input element, for example foot switch 618.

According to some exemplary embodiments, the control unit 604 comprises a motor which is configured to generate negative pressure, for example vacuum forces. In some embodiments, the isolation flow path 603 is connected to the motor 616 via a connector 607.

According to some exemplary embodiments, a user of the system inserts a needle into an ovary, towards an area with a plurality of follicles. Optionally, the user inserts the needle into the ovary under the guidance of Ultrasound imaging. In some embodiments, when the distal end of the needle penetrates into a selected follicle, the user presses the foot switch, for example to activate the motor 616. In some embodiments, the motor generates negative pressure within the isolation flow path 603, within the storage compartment 623 and within the tube 625 leading to the needle 624. In some embodiments, the follicle content including the oocyte is isolated through the tube 625 into the storage compartment 623, while negative pressure is applied.

According to some exemplary embodiments, the system comprises at least one pressure sensor, for example pressure sensor 620 mounted on the isolation flow path. In some embodiments, the pressure sensor 620 monitors changes in pressure during the removal of the follicle content. In some embodiments, the control unit 604 monitors the changes in pressure within the isolation flow path based on signals received from the at least one pressure sensor 620. In some embodiments, when the follicle is empty, a pressure change is measured by the control unit 604 and an indication is delivered to the user by the interface of the control unit 604, for example by speaker 608, screen 612 and/or light indicator 610.

According to some exemplary embodiments, when the follicle is empty, the control unit 604 signals the cartridge 630 to move, for example to replace the follicle storage compartment 603 with a different storage compartment, for example storage compartment 628. Optionally, the control unit 604 activates a motor, for example motor 632 which is connected by an axial shaft to the cartridge, to rotate and to move the cartridge 630. In some embodiments, the cartridge 630 rotates and aligns a new storage compartment with the sealing cover 622.

### Exemplary Multi-Channel Manifold

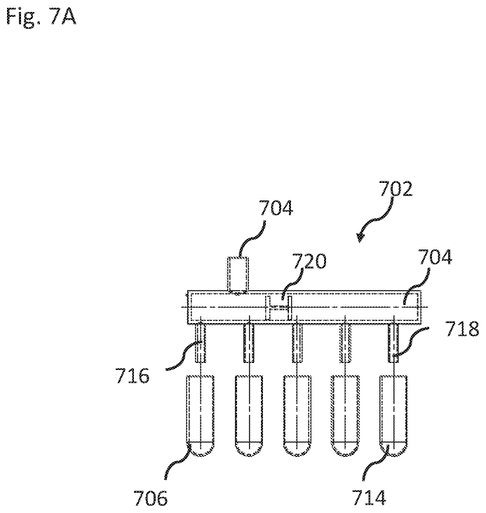

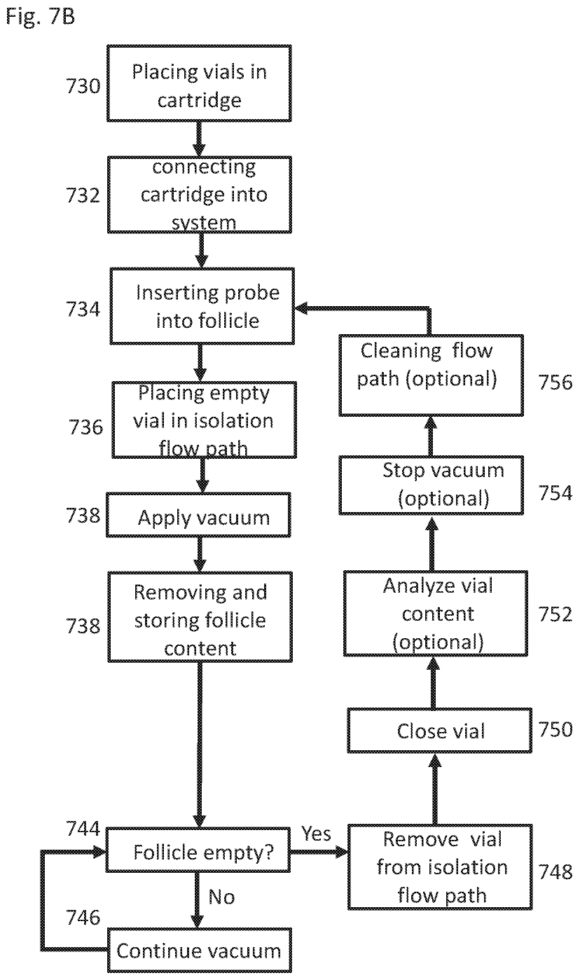

Reference is now made to FIG. 7A, depicting a multi-channel manifold, according to some exemplary embodiments of the invention.

According to some exemplary embodiments, manifold 702 comprises a central lumen, with optionally a single inlet 704, and a plurality of channels for example channels 716 and 718. In some embodiments, a movable flow director 720 is positioned within the lumen, and directs fluid flow from the inlet 704 towards a single channel out of the plurality of channels.

According to some exemplary embodiments, the manifold 702 is shaped and sized to be placed on top of an array of storage compartments, for example vials 706 and 714, where each of the plurality of channels is placed within a vial. Optionally, the organization of at least part of the channels in the manifold matches the organization of the vials in the array.

According to some exemplary embodiments, a control unit of an isolation system moves and aligns the movable flow director with a new channel when a follicle is empty or when a needle penetrates into a new follicle, for example to collect the content of each follicle which comprises an oocyte and FF in a separate vial of the array of vials.

### Exemplary Process for Storage Compartment Replacement

Reference is now made to FIG. 7B depicting a process for storage compartment replacement, according to some exemplary embodiments of the invention.

According to some exemplary embodiments, a plurality of storage compartments, for example vials are placed in a cartridge at 730. In some embodiments, the cartridge is a circular cartridge or a linear cartridge. Optionally, the storage compartments are open when they are placed in the cartridge.

According to some exemplary embodiments, the cartridge is connected to the system, for example system 602 shown in FIG. 6 or system 202 shown in FIG. 2, at 732. In some embodiments, the cartridge is electrically connected to a control unit of the system.

According to some exemplary embodiments, at least part of a follicle probe, for example a needle is inserted into a follicle at 734.

According to some exemplary embodiments, an empty vial is placed in an isolation flow path at 736. In some embodiments, the empty vial is aligned with a sealing cover, for example sealing cover 622 shown in FIG. 6, and is attached to the cover, for example to form a sealed flow path between the needle and the storage compartment. In some embodiments, the sealing cap is advanced and lowered to a stationary storage compartment. Alternatively, the storage compartment is advanced and elevated to interact with a stationary sealing cover, for example to form a sealed flow path between the needle and the storage compartment.

According to some exemplary embodiments, when a sealed flow path is generated, vacuum is applied at 738. In some embodiments, vacuum is applied by activating a vacuum source, fluidically connected to the sealing cover or to the storage compartment.

According to some exemplary embodiments, the content of a follicle is removed and stored at 738. In some embodiments, if the follicle is not empty at 744, then vacuum application is continued at 746. In some embodiments, if the follicle is empty, the vial is removed from the isolation flow path at 748. In some embodiments, the vial is closed at 750. Optionally the vial is closed automatically, for example by placing a cover on the vial in a response to a signal from a control unit of the system. Alternatively, a user receives an indication from the system and manually closes the vial at 750.

According to some exemplary embodiments, the content of the vial is analyzed at 752. In some embodiments, the content of the vial is analyzed optically, by analyzing light delivered through the vial, optionally when the vial is closed. Alternatively, the content of the vial is analyzed by collecting and analyzing some FF stored together with an oocyte within the vial. In some embodiments, the content of the vial is analyzed optically, biologically, chemically and/or genetically.

According to some exemplary embodiments, when the flow path is not sealed, for example when the sealing is not interacting with a vial, vacuum is stopped at 754.

According to some exemplary embodiments, the flow path is cleaned at 756. In some embodiments, the flow path is cleaned by introducing flushing fluid into the isolation flow path, optionally during a short flushing cycle of at least 4 seconds. Optionally, during the flushing cycle a waste storage compartment is connected to the sealing cover.

According to some exemplary embodiments, when flushing is over, the needle is advanced into a new follicle at 734.

### Exemplary Lead Screw Vacuum Sealing Mechanism

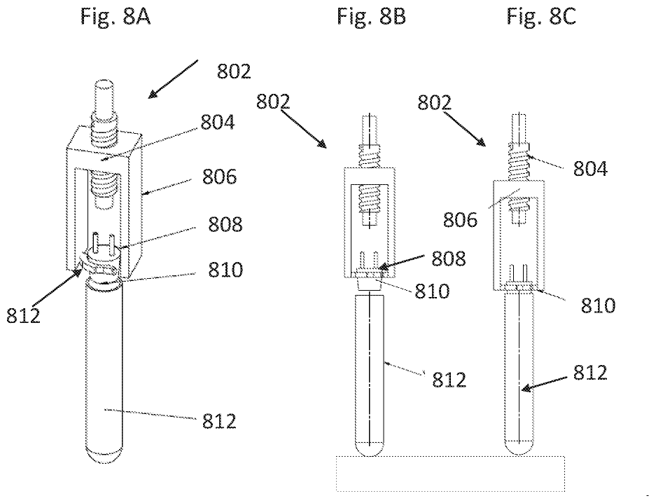

Reference is now made to FIGS. 8A-8C depicting a lead screw vacuum sealing mechanism, according to some exemplary embodiments of the invention.

According to some exemplary embodiments, a lead screw vacuum sealing mechanism 802 comprises a lead screw 804 which penetrates through a lead screw nut 806. In some embodiments, the lead screw nut is part of a structure, for example a frame or an extension that comprises an optionally adjustable sealing cover holder 812, which is configured to hold the sealing cover 808. In some embodiments, the lead screw 804 is functionally connected to a motor that rotates the lead screw, while keeping the lead screw axially stationary. In some embodiments, the lead screw rotation axially moves the lead screw nut up and down, optionally causing axially movement of the sealing cover 808.

In some embodiments, for example as shown in FIG. 8B, the sealing cover 808 is placed over an opened vial 812. In some embodiments, a control unit of an isolation system signals a motor functionally connected to the lead screw 804 to rotate. In some embodiments, the rotation of the lead screw 804 axially moves the sealing cover 808 into an opening of the vial 812. In some embodiments, when the sealing cover 808 moves into the vial 812, a sealing portion 810 of the sealing cover 808 contacts a surface of the vial with sufficient friction to cause sealing of the vial. In some embodiments, rotation of the screw lead in an opposite direction releases the sealing cover 808 from the vial 812 and opens the seal between the sealing portion 810 and the vial 812.

According to some exemplary embodiments, when the sealing cover seals the storage compartment, a suction tube connected to the sealing cover generates negative pressure or vacuum forces sufficient to attract the content of a follicle into the sealed vial.

### Exemplary Linear Vial Cartridge

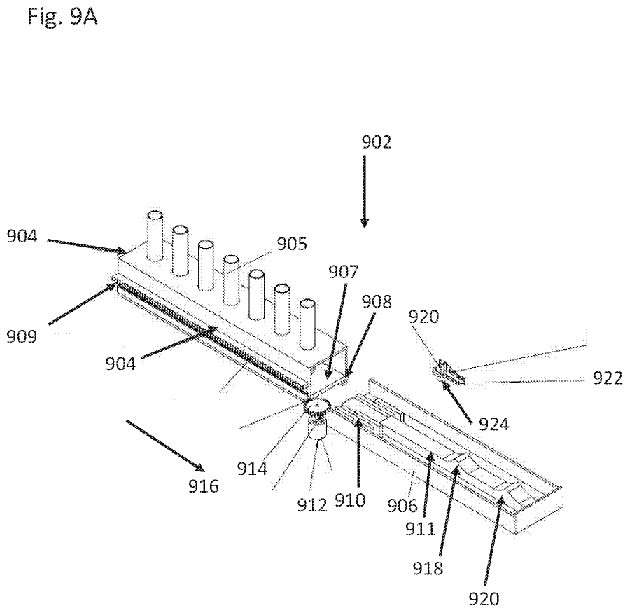

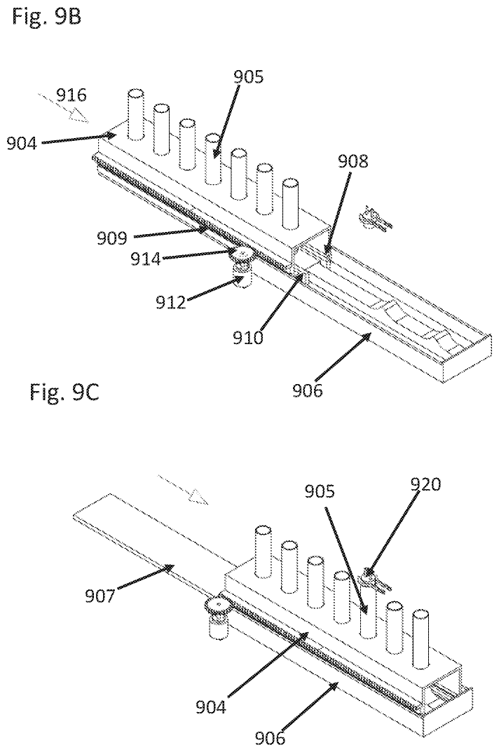

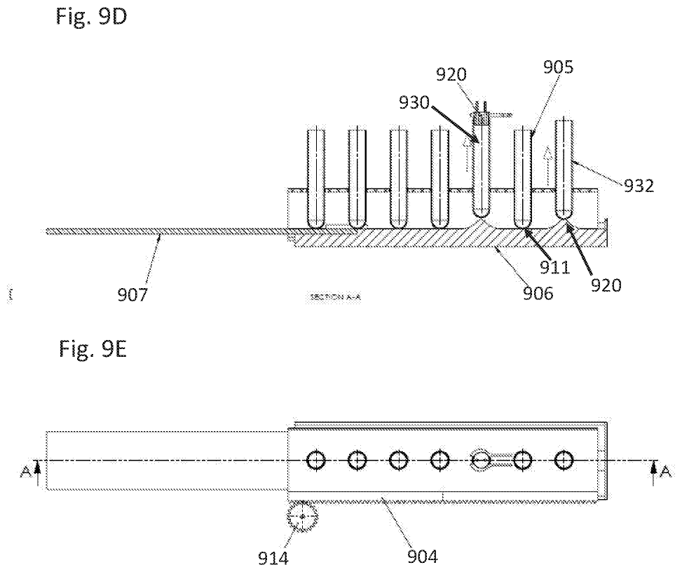

According to some exemplary embodiments, vials are loaded into a cartridge prior to an egg retrieval process, for example to allow isolation and storage of follicular content from a plurality of follicles in separated storage compartments. Reference is now made to FIGS. 9A-9E depicting a linear cartridge of vials, according to some exemplary embodiments of the invention.

According to some exemplary embodiments, a linear cartridge 902 comprises a movable vials rack 904 and a stationary base 906. In some embodiments, vials, for example vial 905 are loaded into openings in the rack 904 until the base of the vial 905 is supported by a flat rack base 907, optionally a detachable rack base. In some embodiments, slots 908 at the bottom of the rack 904 complement protrusions, for example protrusions 910 in the stationary base. In some embodiments, sliding the protrusions 910 within the slots 908 allow for example to connect the rack 904 and the stationary base 906.

According to some exemplary embodiments, the stationary base 906 comprises a flat bottom 911 with at least one bump, shaped and sized to allow elevation of a vial, for example to allow a sealed connection between a dealing cover of an isolation system and an opening of the vial. Optionally, at least one additional bump, for example bump 920 is positioned at a distance from the first bump, for example to allow elevation of the vial to a closing mechanism configured to close the vial with a fixed cover.

According to some exemplary embodiments, a motor 912 positioned near and/or in contact with the stationary base 906 rotates a pinion 914 which matches teeth in a linear gear bar 909 axially positioned along at least one side of the rack 904. In some embodiments, rotation of the pinion 914 within the linear gear bar 909, for example a shown in FIG. 9B loads the rack and the vials into the stationary base 906, and allows the vials to slide along the flat bottom 911. In some embodiments, the flat rack base 907 is detached from the rack 904 with the vials when the rack 904 and the vials is loaded onto the stationary base 906.

According to some exemplary embodiments, for example as shown in FIG. 9C when vial 905 reaches bump 918, the vial is elevated towards a stationary sealing cover, for example a sealing cover 920 or sealing cover 808 shown in FIG. 8A. In some embodiments, the stationary sealing cover 920 is fixedly aligned by a clipper 922 above the bump 918. In some embodiments, for example when the follicle is empty, the pinion rotates and lowers the vial 905 from the bump, causing the sealing cover 920 to detach from the vial 905 opening. Optionally, the pinion continues to rotate and moves a new vial on the bump causing the vial to contact the sealing cover, for example when the needle penetrates into a new follicle.

According to some exemplary embodiments, when the pinion rotates, a new vial 930 is elevated towards the sealing cover 920, while the previous vial 905 is lowered to the flat bottom 911. In some embodiments, by further rotation of the pinion, a vial can be elevated on bump 920 for example to be closed with a cover by a closing mechanism positioned above the bump 920.

### Exemplary Round Vial Cartridge

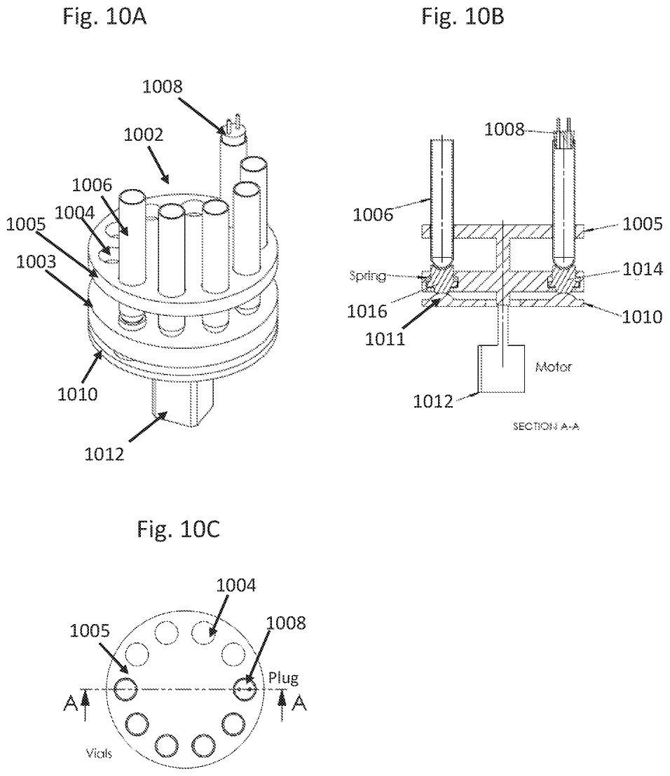

Reference is now made to FIGS. 10A-10C depicting a round vial cartridge, according to some exemplary embodiments of the invention.

According to some exemplary embodiments, a round vial cartridge 1002 comprises an upper round plate 1005 having a plurality of openings 1004, shaped and sized to allow insertion of vials, for example vial 1006 at least partly through the openings 1004. Additionally, the cartridge comprises a lower base plate, optionally having a plurality of movable portions 1014, each movable portion is aligned underneath a bottom of a vial. In some embodiments, the movable portion is configured to move upwards and to elevate a vial. In some embodiments, the movable portion comprises a spring that keeps the vials down as a default position.

According to some exemplary embodiments, the cartridge is placed on a stationary plate 1010 which comprises at least one bump, and is mechanically connected to a motor 1012, optionally through a drive shaft. In some embodiments, the motor rotates the round cartridge relative to the stationary plate 1010.

According to some exemplary embodiments, rotation of the cartridge 1002, moves a movable portion over a bump, which pushes the movable portion up, optionally against the force applied by the spring. In some embodiments, the movable portion pushes the vial against a stationary sealing cover, for example sealing cover 1008 aligned with an opening of the vial. In some embodiments, when a follicle is empty or when the needle penetrates into a new follicle, a control unit of a system signals the motor 1012 to rotate the cartridge, for example to lower and detach an existing vial filled with follicle content and to elevate an empty vial towards the sealing cover.

### Exemplary Sealing Cover Releasing Mechanism

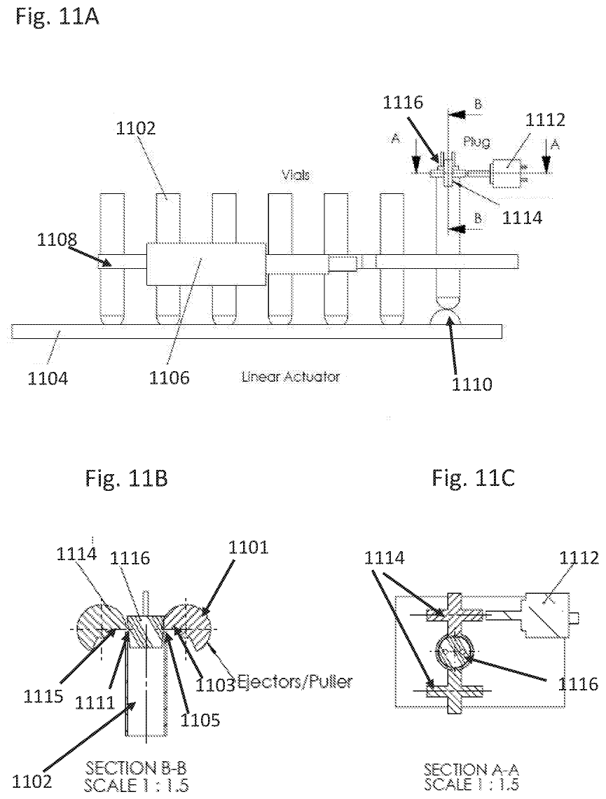

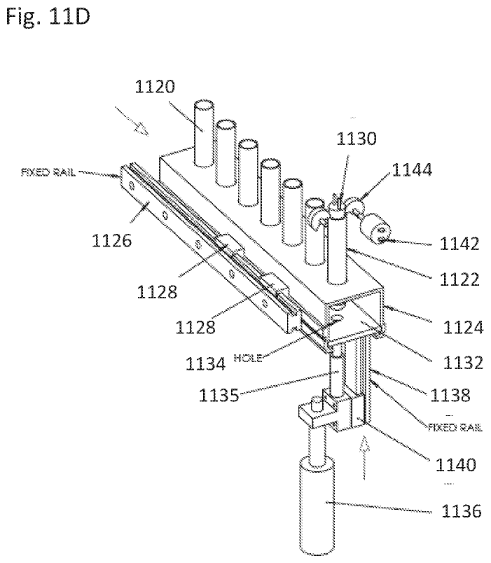

Reference is now made to FIGS. 11A-11C depicting a motorized sealing cover releasing mechanism, according to some exemplary embodiments of the invention.

According to some exemplary embodiments, vials, for example vial 1102 are positioned inside a linear vial cartridge 1108. In some embodiments, the cartridge is axially advanced by an actuator 1106 on a stationary base 1104, which includes a bump 1110. In some embodiments, when a vial reaches bump 1110, the vial is elevated towards sealing cover 1116. In some embodiments, when the vial is elevated at least one wheel 1114, optionally a silicon wheel is rotated. Alternatively, the vial is pushed against the sealing cover 1116 without rotating the wheel 1114. In some embodiments, the wheel 1114 is rotated passively, when the vial is pushed up. Optionally, the wheel is rotated until it reaches a pre-determined position.

According to some exemplary embodiments, in order to detach the vial from the sealing cover 1116, for example when a follicle is empty and the control unit signals the cartridge to replace the vial with an empty one, a motor 1112 functionally connected to the wheel 1114 rotates and moves the wheel. In some embodiments, an outer surface of the wheel 1114 applies friction forces on the vial.

According to some exemplary embodiments, for example as shown in FIG. 11B the at least one wheel 1114 is partly round, and comprises at least one flat surface 1111, shaped and sized to contact part of an upper surface 1111 of the vial. Optionally, at least one additional wheel, for example wheel 1101 is positioned in an opposite side of the vial 1102, and optionally in parallel to the wheel 1114. In some embodiments, the wheel 1114 is partly round and comprises at least one surface 1103, shaped and sized to contact part of an upper surface 1105 of the vial 1102. In some embodiments, the motor 1112 rotates the two wheels 1101 and 1114, optionally simultaneously, in opposite directions. In some embodiments, rotation of the wheels 1101 and 1114 in opposite directions applies friction force on the upper surfaces 1105 and 1111, pushing the vial down and away from the sealing cover 1116.

Exemplary Active Attachment of a Storage Compartment into a Sealing Cover

According to some exemplary embodiments, a storage compartment, for example a vial placed in a movable rack, is forwarded to a selected axial position below a stationary sealing cover. In some embodiments, a movable pusher is elevated by a motor through an opening in the base of the rack, and pushes the bottom of the vial upwards towards the sealing cover. In some embodiments, when a follicle is empty and/or when a needle of the isolation system penetrates into a new follicle, an active releasing mechanism, for example the mechanism described in FIGS. 11A-11C pushes the vial down, towards the base of the rack, optionally simultaneously with the retraction of the pusher. Reference is now made to FIG. 11D depicting a device or active attachment of a sealing cover to an opening of a storage compartment, according to some exemplary embodiments of the invention.

According to some exemplary embodiments, at least one storage compartment, for example vials 1120 and 1122 are positioned within a cartridge 1124. In some embodiments, the bottom of vials 1120 and 1122 is supported by the cartridge base 1132. In some embodiments, the cartridge is connected to a fixed rail 1126 by guides 1128 connected to the cartridge 1124. In some embodiments, the guides are configured to axially slide along the rail 1126, as the cartridge advances.

According to some exemplary embodiments, the cartridge base 1132 comprises a plurality of openings, for example opening 1134, optionally aligned underneath each vial. In some embodiments, the openings diameter or largest axis is smaller than a diameter of the vial, for example to support the vial base. In some embodiments, a size of the largest dimension of opening 1134, for example the diameter of the opening 1134 is smaller than 10 mm, for example 5 mm, 4 mm, 3 mm or any intermediate smaller or larger value. In some embodiments a size of the largest dimension, for example the diameter of the storage compartment, is at least 5 mm, for example 5 mm, 7 mm, 8 mm, 10 mm, 12 mm or any intermediate, smaller or larger value.

According to some exemplary embodiments, a pusher 1135 has a diameter smaller than the diameter of the opening 1134 and is configured to move along an axis perpendicular to the cartridge base 1132 and through the openings. In some embodiments, a motor 1136 is mechanically connected to the pusher 1135, and axially moves the pusher 1135 through the openings. In some embodiments, the pusher and the motor are stationary relative to the movable cartridge, by a fixed rail 1138.

According to some exemplary embodiments, a control unit of an isolation system, for example system 202 or system 602, signals the cartridge 1124 to move to a position where a vial, for example vial 1122 is positioned underneath a stationary sealing cover 1130. In some embodiments, when the vial is in the desired position, the motor 1136 is activated and pushes the pusher 1135 through the opening 1134 towards the base of the vial 1122. In some embodiments, the pusher 1135 pushes the vial 1122 towards the sealing cover 1130 with sufficient force to form a vacuum sealing between the sealing cover 1130 and the vial 1122. In some embodiments, throughout the isolation process of follicular content into the vial 1122, the pusher 1135 applies force against the sealing cover 1130, for example to maintain the sealing between the cover and the vial.

According to some exemplary embodiments, in order to replace a vial, the control unit activates a releasing mechanism rotor 1142, which rotates and pushes the vial down and away from the sealing cover, for example to break the sealing between the sealing cover 1130 and the vial 1122. Optionally, the control unit signals the motor 1136 to retract the pusher 1135, for example by deactivating the motor 1136 or by signaling the motor 1136 to rotate in an opposite direction to the rotation direction used to elevate the pusher.

### Exemplary Multi-Axis Storage Compartment Attachment to Sealing Cover

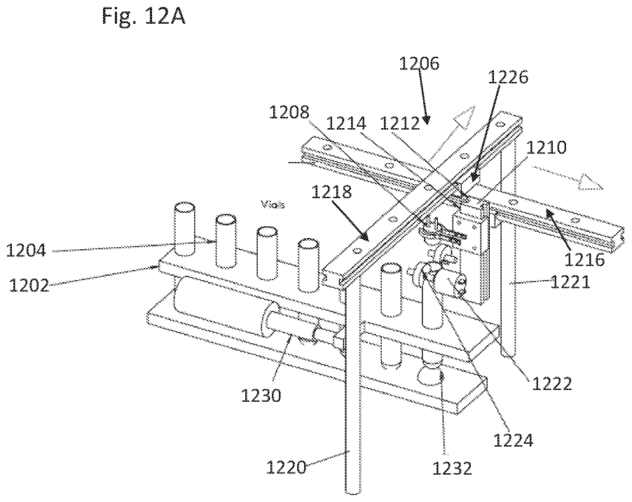

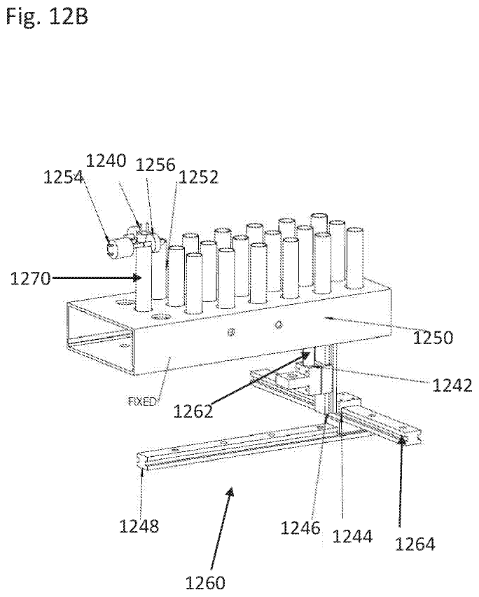

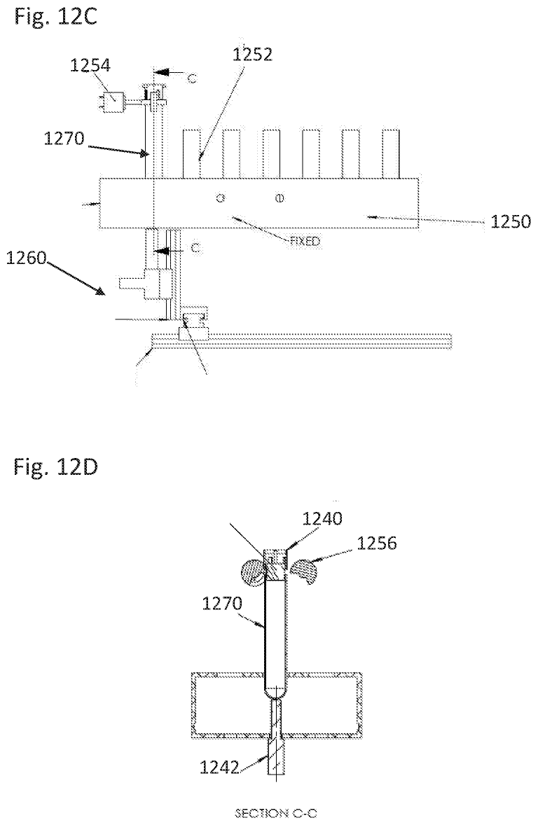

Reference is now made to FIG. 12A depicting a gantry system for attaching a sealing cover to an opening of a vial, according to some exemplary embodiments of the invention.

According to some exemplary embodiments, the sealing cover is mounted on an xyz axis rail system that moves above a cartridge of vials. In some embodiments, xyz axis rail system allows, for example to move the sealing cover to a selected vial, and to optionally close the vial by lowering the sealing cover along the z axis into the vial.

According to some exemplary embodiments, a multi-axis system 1206, comprises a sealing cover 1208 mounted on a z-axis rail 1214 through a movable z-axis guide 1212, configured to move the sealing cover 1208 along the z-axis rail. In some embodiments, the z-axis rail 1214 is connected to an x-axis rail 1216 via an x-axis guide 1210, configured to move the z-axis rail 1214 along the x-axis rail 1216. In some embodiments, the x-axis rail is movably connected to a y-axis rail 1218 mounted between two posts 1220 1221. In some embodiments, the x-axis rail 1216 is movably connected to the y-axis rail 1218 by y-axis guide 1226.

According to some exemplary embodiments, the y-axis rail is mounted over a cartridge 1202 which includes at least one vial, for example vial 1204. In some embodiments, the multi-axis system 1206 moves the sealing cover 1208 above a selected vial in cartridge 1202. In some embodiments, the system comprises at least one optical sensor and/or at least one encoder, for example to determine the exact position of the sealing cover, optionally relatively to the cartridge 1202 and/or relatively to a selected vial within the cartridge.

According to some exemplary embodiments, the sealing cover 1208 moves along the z-axis rail and is attached to the vial opening with sufficient force to seal a passage between the sealing cover and the vial. In some embodiments, to release the sealing cover from the vial opening, a releasing mechanism, for example a releasing mechanism show in FIGS. 11A-11D is used. In some embodiments, a motor 1222 of the releasing mechanism rotates at least one wheel 1224 to push the vial down and away from the sealing cover 1208. Optionally, bump 1232 elevates the tube/vial, for example to bring the tube/vial for a selected position for collecting the FF. In some embodiments, the sealing cover is connected to an XY-system, where the sealing cover moves by an x-axis rail and a y-axis rail. In some embodiments, in an XY-system, the cartridge moves to place a vial over a bump 1232 in a fixed cartridge base. In some embodiments, the vial is elevated towards the sealing cover, for example as shown in FIG. 9A. In some embodiments, the sealing cover is released from the vial opening, for example by a releasing mechanism as described above.

According to some exemplary embodiments, the XY-system and/or the XYZ-system comprise at least one optical sensor and/or at least one encoder to determine the position of the sealing cover. Optionally, the XY-system and/or the XYZ-system comprises a vial closure mechanism, configured to place a cover over an opening of a vial after the release of the sealing cover from the vial opening.

According to some exemplary embodiments, a sealing cover, for example sealing cover 1240 is connected to an XYZ-system 1260, for example as shown in FIGS. 12B-12D. In some embodiments, the system 1260 comprises a pusher 1242, shaped and sized to be pushed by a motor through holes in a cartridge base, for example as described in FIG. 11D. In some embodiments, the pusher 1242 is movable connected to z-rail 1262, and is configured to move up and down along the z-rail 1262. In some embodiments, the z-rail 1262 is movably connected to an x-rail 1264, and is configured to move along the x-rail 1264. In some embodiments, the x-rail 1264 is movably connected to a y-rail 1248, and is configured to move along the y-rail 1248.

According to some exemplary embodiments, the sealing cover 1240 is fixedly mounted to the z-rail 1262, in parallel to the pusher 1262. In some embodiments, the xyz-system moves around a cartridge 1250 to a selected vial, for example vial 1270. In some embodiments, the sealing cover is placed at a fixed position above the cartridge 1250 and the vials in the cartridge. In some embodiments, when reaching a selected open vial, for example vial 1270 the sealing cover 1240 is positioned above the vial 1270 and the pusher 1242 moves upwards along the z-rail and pushes the vial towards the sealing cover. In some embodiments, in order to release the sealing cover 1240 from the vial 1270, a releasing mechanism which comprises a motor 1254 connected to at least one wheel 1256, pushes the vial down, optionally, while retracting the pusher 1242.

### Exemplary Optical Analysis Assembly

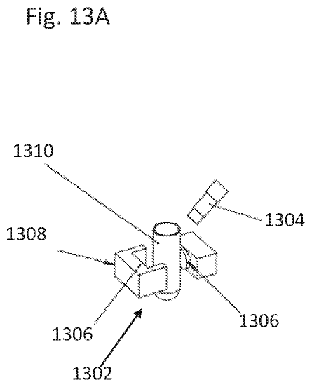

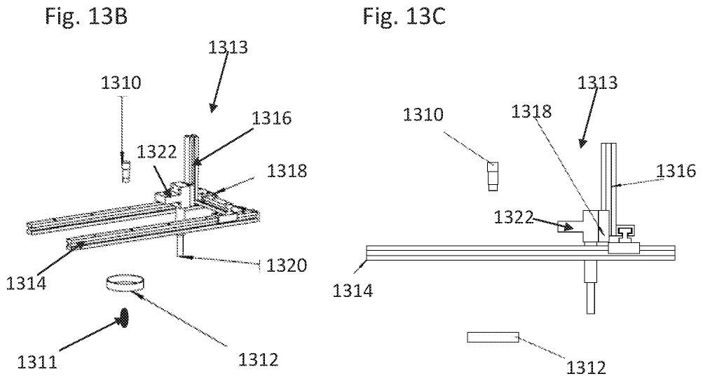

Reference is now made to FIG. 13A, depicting an optical analysis assembly adjacent to a storage compartment, according to some exemplary embodiments of the invention.

According to some exemplary embodiments, an optical analysis assembly, for example optical analysis assembly 1302 is placed near a storage compartment, for example vial 1310. In some embodiments, the optical assembly 1302 comprises a photo coupler 1306 configured to couple a light source, for example LED 1306 to the vial, for example to allow illumination of the vial content by light emitted from the LED 1306. In some embodiments, the optical assembly 1302 comprises a photodiode 1306 or an optical sensor positioned in a light path crossing the vial 1310. In some embodiments, the optical assembly 1302 comprises a camera 1304, configured to capture and record light travelling through the vial 1310.

According to some exemplary embodiments, for example as shown in FIGS. 13B and 13C the optics assembly is stationary, and the vials are advanced for analysis by the optics assembly, optionally using a multi-axis system.

According to some exemplary embodiments, for example a shown in FIGS. 13B and 13C, the optics assembly is stationary and comprises a sensor and/or a camera 1310. Optionally, the optics assembly comprises a lens 1312 and/or an illumination source 1311. In some embodiments, a multi-axis system, for example system 1313 is configured to move and position a storage compartment in a field of view (FOV) of the optics assembly, for example between the camera 1310 and the illumination source 1311. In some embodiments, the system 1313 comprises a movable storage compartment holder, for example a movable vial holder 1322, connected to a z-rail 1316, and configured to move along the z-rail 1316, optionally by a motor or an actuator functionally connected to the vial holder 1322. In some embodiments, the z-rail 1316 is connected to a y-rail 1318, and is configured to move along the y-rail, optionally by a motor or an actuator functionally connected to the z-rail. In some embodiments, the y-rail is connected to an x-rail 1314, and is configured to move along the x-rail, optionally by a motor or an actuator functionally connected to the y-rail. In some embodiments, movement of the vial in optionally 3 dimensions allows to position the vial in the FOV of the optics assembly, and to focus the optics assembly on a desired focal plane by adjustment of the vial height using the z-rail 1316.

According to some exemplary embodiments, the optical assembly is configured to detect one or more of changes in color, color depth and/or level of color, light diffraction, and/or turbidity of the vial content. In some embodiments, at least one optical assembly is positioned along the isolation flow path, the FF fluid path and/or the oocyte path, for example as described in FIG. 2. Alternatively or additionally, at least one optical assembly is positioned near a storage compartment, for example a follicular content storage compartment, FF storage compartment, and/or oocyte storage compartment.

According to some exemplary embodiments, the optical assembly is configured to determine the presence of an oocyte in the isolation flow path, in the oocyte flow path and/or in the oocyte storage.

It is expected that during the life of a patent maturing from this application many relevant follicular isolation systems will be developed; the scope of the term follicular isolation system is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method for isolating and storing follicular content, comprising:
repeatedly collecting follicular content comprising an oocyte and follicular fluid (FF) from a plurality of follicles of a female subject during a single oocyte retrieval process;
storing each oocyte and follicular fluid originating from the same follicle in a separate storage compartment from the rest of said collected follicular content.

2. The method of claim 1, comprising:
isolating a follicular fluid sample from said stored follicular fluid;
associating said follicular fluid sample with a specific oocyte.

3. (canceled)

4. The method of claim 2, wherein said storing comprises storing said follicular content from each follicle of said plurality of follicles in according to a pre-determined order, and wherein said associating comprises associating said FF sample with said oocyte based on said order.

5. The method of claim 2, wherein a volume of said FF sample is at least 10 microliters.

6. The method of claim 2, comprising analyzing said FF sample to determine at least one parameter related to a state of said associated oocyte.

7. The method of claim 6, wherein said at least one parameter comprises oxidation levels.

8. (canceled)

9. The method of claim 6 comprising selecting an oocyte for an IVF process based on results of said FF sample analysis.

10. The method of claim 6, comprising cryopreserving at least some isolated oocytes from said plurality of follicles based on results of said FF sample analysis.

11. A system for sorting follicle content, comprising:
a storage assembly comprising a plurality of storage compartments;
a first fluid flow path between a needle at least partly insertable into a follicle and a first storage compartment of said plurality of storage compartments;
at least one movable portion comprising an actuator configured to individually align a distal opening of said first fluid flow path with a different storage compartment of said storage assembly;
a control unit, comprising:
a control circuitry electrically connected to said actuator and configured to signal said actuator to individually align said distal opening with a different storage compartment of said storage assembly.

12. The system of claim 11, comprising an interface configured to deliver a human detectable indication related to alignment of said distal opening with at least one storage compartment of said plurality of said storage compartments.

13.-14. (canceled)

15. The system of claim 12, wherein said movable portion comprises a movable storage assembly, and is configured to move said storage assembly relative to said distal opening in response to a signal received from said control circuitry.

16. The system of claim 12, comprising at least one sensor on said first flow path electrically connected to said control circuitry, wherein said control circuitry is configured to signal said movable portion to align said distal opening with said different storage compartment of said storage assembly based on signals received from said at least one sensor.

17.-20. (canceled)

21. The system of claim 12, comprising a first negative pressure source functionally connected to said first fluid flow path, and configured to generate negative pressure within said first fluid flow path.

22. The system of claim 21, wherein said control circuitry signals said interface to generate said human detectable indication when said first negative pressure source is activated.

23. (canceled)

24. The system of claim 16 comprising a second fluid flow path connecting said first fluid flow path and at least one follicular fluid (FF) collecting compartment.

25. The system of claim 24, wherein said FF collecting compartment comprises an optical analyzer cuvette.

26. The system of claim 24, comprising a second negative pressure source functionally connected to said second fluid flow path and electrically connected to said control circuitry, wherein said second negative pressure source is configured to generate negative pressure within said second fluid flow path in response to electrical signals received from said control circuitry.

27. The system of claim 26, wherein said control circuitry is configured to signal said second negative pressure source to generate said negative pressure within said second flow path for a time duration sufficient to deliver at least 50 μl of FF into said FF collecting compartment.

28. (canceled)

29. The system of claim 24, comprising a filter positioned within said second fluid flow path, wherein said filter comprises a plurality of pores shaped and sized to prevent passage of an oocyte from said first fluid flow path into said second fluid flow path.

30. The system of claim 29, wherein a largest dimension of said pores is in a range of 2-50 micron.

31.-50. (canceled)
